(19) United States
(12) Patent Application Publication
(10) Pub. No.: US 2019/0010174 A1
(43) Pub. Date: Jan. 10, 2019

(54) Crosslinking Monomers With at Least One Sulfur Atom

(71) Applicant: Kulzer GmbH, Hanau (DE)

(72) Inventors: Andreas Utterodt, Neu Anspach (DE); Helmut Ritter, Wuppertal (DE); Moniralsadat Tabatabai, Duesseldorf (DE); Melanie Nadine Kemnitz, Duesseldorf (DE)

(21) Appl. No.: 15/748,805

(22) PCT Filed: Jul. 29, 2016
(86) PCT No.: PCT/EP2016/068155

§ 371 Date: Jan. 30, 2018

(30) Foreign Application Priority Data
Jul. 31, 2015 (DE) 10 2015 112 602.8

(51) Int. Cl. C07F 9/38; C07F 9/40; C08F 30/02

(52) U.S. Cl. CPC C07F 9/3826; A61K 6/083; C08F 30/02; C07F 9/4009

(57) ABSTRACT

The invention relates to a cross-linking monomer with at least one sulfur atom, representable by a structure of formula (I)

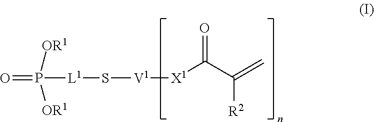

wherein the symbols have the following meaning:
L¹ is a linear, branched or cyclic, saturated or unsaturated, aliphatic or heteroaliphatic group with 2 to 10 carbon atoms; an aromatic or heteroaromatic group with 4 to 12 carbon atoms or a group with 6 to 14 carbon atoms, which comprises at least one saturated or unsaturated, aliphatic and/or heteroaliphatic residue and at least one aromatic and/or heteroaromatic residue, wherein the phosphorus atom is directly bonded with a carbon atom;
V¹ is a saturated or unsaturated, aliphatic or heteroaliphatic group with 2 to 50 carbon atoms; an aromatic or heteroaromatic group with 5 to 50 carbon atoms or a group with 6 to 50 carbon atoms, which comprises at least one saturated or unsaturated, aliphatic and/or heteroaliphatic residue and at least one aromatic and/or heteroaromatic residue;
X¹ is the same or different, O or NR³, each time it occurs;
R¹ is H or a hydrolyzable group;
R² is H or an alkyl residue with 1 to 4 carbon atoms;
R³ is H or an aliphatic, heteroaliphatic, aromatic or heteroaromatic group with 1 to 6 carbon atoms, and
n is 2, 3, 4, 5 or 6,
wherein the sulfur atom in the structure according to formula (I) is separated from group X¹ by a maximum of 12 bonds, which group is spaced from the sulfur atom by the lowest number of bonds, wherein double bonds or triple bonds are counted as a single bond.

The invention relates to a cross-linking monomer with at least one sulfur atom, a composition containing said monomer and the use of said monomer in dental medicine.

Polymerizable phosphonic acids are of polymer chemical and technical significance as co-monomers in particular, and enable the production of organic polymers with improved thermal stability, improved adhesion properties, reduced flammability and improved solubility in polar solvents. In this context, numerous monomer phosphonic acids have been synthesized and polymerized with polymerizable vinyl, dienyl, allyl or styryl groups (cf. Houben-Weyl, Methoden der Organischen Chemie, Vol. E 20 (2nd section), G. Thieme Verlag, Stuttgart-New York 1987, 1300 ff.). Polymerizable phosphonic acids are also known as components of dental adhesives (cf. N. Moszner, U. Salz, J. Zimmermann, Dental Materials 21 (2005) 895-910) and as the subject of numerous patents and patent applications, e.g. DE 100 18 968 C1, DE 102 34 326 B3, DE 199 18 974 A1, EP 1 057 468 A1 and EP 1 169 996 A1.

Patent document WO 2013/083734 A1 describes polymerizable bisphosphonic acids, which can be integrated into a polymer matrix in a similar manner to the monomers described above. Sulfurous monomers are not explicitly named in WO 2013/083734 A1. In a very generalized embodiment, which is not described as a preferred embodiment, sulfur atoms can be present in chains, for example as thioether group —S—, which bonds at least one polymerizable group with an (m+2)-fold substituted aliphatic C₁-C₈ residue, which in WO 2013/083734 A1 is depicted with symbol A. The residue A comprises no sulfur atoms and is bonded with two phosphonic acid groups. Between the two phosphonic acid groups, no sulfur atoms can therefore be arranged in monomers according to WO 2013/083734 A1. Further, group A represents a branch point, since all polymerizable groups bond to group A. There is therefore no embodiment in which a sulfur atom is arranged between a phosphorus atom and a branch point.

Further, there is no specific reference to monomers containing sulfur atoms. The embodiment described is far more selected from a long list of groups which can be present in a chain, which bonds a polymerizable group with the residue A.

The monomers described above lead to serviceable dental materials. However, there is a constant need for improving the properties of these materials. In particular, the adhesion of these materials to different substrates which are of importance in dental medicine should be improved. Further, the formulation of compositions for producing these dental materials should be simplified. A further object can be regarded as designing the handling of the formulations usable in dental medicine in such a way that it becomes simpler. Additionally, the hardening of polymerizable compositions should be improved. Furthermore, the mechanical properties of polymerizable dental materials should be increased.

These and other objects not explicitly described, but which can easily be derived and extrapolated from the contexts discussed here in the introduction, are attained by means of a monomer comprising at least one sulfur atom with all the features of Claim 1. Advantageous modifications of the monomers according to the invention are protected in the dependent claims which refer to Claim 1.

The subject of the present invention is therefore a networked monomer, representable by a structure of formula (I)

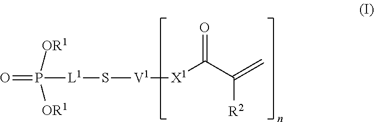

wherein the symbols have the following meaning:

L¹ is a linear, branched or cyclic, saturated or unsaturated, aliphatic or heteroaliphatic group with 2 to 10 carbon atoms; an aromatic or heteroaromatic group with 4 to 12, preferably 5 to 10 carbon atoms or a group with 6 to 14 carbon atoms, which comprises at least one saturated or unsaturated, aliphatic and/or heteroaliphatic residue and at least one aromatic and/or heteroaromatic residue, wherein the phosphorus atom is directly bonded with a carbon atom;

V¹ is a saturated or unsaturated, aliphatic or heteroaliphatic group with 2 to 50 carbon atoms; an aromatic or heteroaromatic group with 5 to 50 carbon atoms or a group with 6 to 50 carbon atoms, which comprises at least one saturated or unsaturated, aliphatic and/or heteroaliphatic residue and at least one aromatic and/or heteroaromatic residue;

X¹ is the same or different, O or NR³, each time it occurs;

R¹ is H or a hydrolyzable group;

R² is H or an alkyl residue with 1 to 4 carbon atoms;

R³ is H or an aliphatic, heteroaliphatic, aromatic or heteroaromatic group with 1 to 6 carbon atoms, and

n is 2, 3, 4, 5 or 6,

which is characterized in that

the sulfur atom in the structure according to formula (I) is separated from group X¹ by a maximum of 12 bonds, which group is spaced from the sulfur atom by the lowest number of bonds, wherein double bonds or triple bonds are counted as a single bond.

The monomers according to the invention with at least one sulfur atom are particularly suited for applications in dental medicine, and have a good adhesive capacity, in particular in relation to tooth hardening substance and/or metals used in dental medicine.

The monomers according to the invention with at least one sulfur atom preferably have a very good adhesion capacity on solid surfaces when in a hardened state in products, preferably in relation to Dentin, here with an adhesive force of preferably at least 12.5 MPa.

The monomers according to the invention with at least one sulfur atom are suitable as additives for and as a component of adhesion improving adhesive(s), in particular suitable for mineral substrates, and particularly for dental hardening materials. They also enable application in the non-etch method, since the bonds according to the invention, including in combination with one or more additional monomers, evidently lead to the surface modification required for the bonding procedure.

Furthermore, the monomers according to the invention according to formula (I) show an adhesion-improving effect as a component of dental filling composites and fixing cements, lining materials, flow materials, fissure sealers, lacquers, root canal materials, core build-up materials and temporary repair materials (in particular, inlays, onlays, crowns, bridges and/or fixing materials).

Here, the monomers with at least one sulfur atom according to the invention preferably, when in a fully hardened state, lead to products with excellent mechanical properties and a high level of durability. This entails high abrasion resistance (wear resistance) and high stability under loads (flexural strength). Further, the monomers according to the present invention preferably lead to a lower or reduced absorption of water or solubility.

Furthermore, the monomers with at least one sulfur atom according to the invention can be processed very well, wherein this is reflected in a very good level of solubility in different solvents and relatively good, rapid polymerization.

The above advantages can be realised in full or partially. In general, the monomers are distinguished by improved adhesion on different materials.

The term “cross-linking monomer” describes a monomer which has at least two radically polymerizable groups, preferably (meth)acrylate groups. (Meth)acrylate groups are groups derived from acrylic acid, methacrylic acid, acrylamide or methacrylamide, for example.

Adjacent carbon atoms in the sense of the present invention are carbon atoms which are directly linked to each other. Further, “adjacent residues” means in terms of the definition of the residues that these residues are bonded with the same carbon atom or to adjacent carbon atoms. These definitions apply accordingly to the terms “adjacent groups” and “adjacent substitutes”, among others. The term “non-adjacent” carbon atoms results from this definition in a complementary manner.

The term linear, branched or cyclic, saturated or unsaturated, aliphatic or heteroaliphatic group with 2 to 10 carbon atoms is known in the specialist field and describes a group with 2 to 10 carbon atoms, which comprises no aromatic or heteroaromatic group between the at least two bonding points of the respective group, and preferably comprises no aromatic or heteroaromatic group. Thus, for example, group L¹ has at least one bonding point to the phosphorus atom and at least one bonding point to the sulfur atom. The term “no aromatic or heteroaromatic group between the at least two bonding points” means that no aromatic or heteroaromatic group participates in the bonds between the phosphorus atom and the sulfur atom, although it may well be present as a side group or a substituent.

Saturated aliphatic groups which are suitable as group L¹ in formula (I) include, among others, ethylene, 1,2- and 1,3-propylene, 1,2, 1,3- and 1,4-butylene, pentylene, hexylene, heptylene, octylene, nonylene, decylene, undecylene, dodecylene and tetradecylene.

Unsaturated aliphatic groups which are suitable as group L¹ in formula (I) differ from the groups described above due to the presence of at least one C—C double or triple bond. These include, among others, ethenylene, 1,2- and 1,3-propenylene, 1,2, 1,3- and 1,4-butenylene, pentenylene, hexenylene, heptenylene, octenylene, nonenylene, decenylene, undecenylene, dodecenylene and tetradecenylene.

As well as carbon and hydrocarbon atoms, heteroaliphatic groups have heteroatoms such as nitrogen, oxygen, phosphorous, silicon and sulfur atoms. Preferred heteroaliphatic groups, which can be saturated or unsaturated, are derived among others from the saturated and unsaturated aliphatic groups described above, wherein one or more non-adjacent CH₂ groups are substituted with —(C═O)—, —(C═S)—, —(C═NR³)—, —C(═O)O—, —C(═O)NR³—, —NR³—, P(═O)(R³), —[CH(CxH2x)P(═O)(OR¹)₂]—, —O—, —S—, —(SO)— or (—SO₂)—, wherein R³ is H or an aliphatic, heteroaliphatic, aromatic or heteroaromatic group with 1 to 6 carbon atoms, x is an integer in the range of 0 to 6, preferably 1 to 5.

Further, the symbol L¹ can stand for an aromatic or heteroaromatic group with 4 to 12, preferably 5 to 10 carbon atoms. An aromatic or heteroaromatic group comprises at least one aryl or heteroaryl group, which is respectively bonded with the adjacent group. In the case of group L¹, the sulfur and the phosphorous atom are respectively bonded with one atom of the aromatic system, preferably to a phenylene group.

An aryl group in the sense of this invention contains 6 to 12 C-atoms; a heteroaryl group in the sense of this invention contains 4 to 12, preferably 5 to 10 C-atoms and at least one heteroatom, with the proviso that the total of C-atoms and heteroatoms is at least 5. The heteroatoms are preferably selected from N, O and/or S. Here, an aryl group or heteroaryl group is understood as being either a single aromatic cyclus, i.e. benzol, or a single heteroaromatic cyclus, such as pyridine, pyrimidine, thiopene, etc., or a condensed aryl or heteroaryl group, such as naphthalene, quinoline, isoquinoline, etc. The aryl or heteroaryl group can here be substituted by one or more linear, branched or cyclic, saturated or unsaturated, aliphatic or heteroaliphatic groups, with the proviso that the respective total number of C-atoms for an aromatic or heteroaromatic group is not exceeded.

Further, group L¹ can stand for a group with 6 to 14 carbon atoms, which comprises at least on saturated or unsaturated, aliphatic or heteroaliphatic residue and at least one aromatic and/or heteroaromatic residue. This group is a combination of a linear, branched or cyclic, saturated or unsaturated, aliphatic or heteroaliphatic group and an aromatic or heteroaromatic group, as described above. Here, between the at least two bonding points, the group can have at least one aromatic or heteroaromatic group and at least one saturated or unsaturated, aliphatic and/or heteroaliphatic group. Further, a linear, branched or cyclic, saturated or unsaturated, aliphatic or heteroaliphatic group, which is present between the at least two bonding points, can be substituted by one or more aromatic and/or heteroaromatic residues.

In a particularly preferred embodiment, the symbol L¹ can represent a linear, branched or cyclic alkylene group with 2 to 10 carbon atoms, in which one or more non-adjacent CH₂ groups can be substituted with —R³C═CR³—, —C≡C—, —(C═O)—, —(C═S)—, —(C═NR³),—, —C(═O)O—, —C(═O)NR³—, —NR³—, P(═O)(R³), —[CH(CₓH₂ₓ)P(═O)(OR¹)₂]—, O—, —S—, —(SO)— or (—SO₂)—, wherein R³ has the meaning described above for formula (I) and x is an integer in the range of 0 to 6, preferably 1 to 5. If the group L¹ has a phosphorous atom, this phosphorous atom is preferably spaced by at least 4, particularly preferred by at least 6 bonds from the phosphorous atom shown in formula (I). In a further embodiment, the second phosphorous atom can be arranged closer to the sulfur atom than the first phosphorous atom. Arranged closer means that the number of atoms present between the sulfur atom and the phosphorous atoms differs.

In a particularly preferred manner, L¹ is a linear, branched or cyclic alkylene group with 2 to 10, preferably 3 to 5 carbon atoms, which has no heteroatoms. In a particularly preferred manner, group L¹ is a saturated hydrocarbon residue with 2 to 6, preferably 3 to 5 carbon atoms, which comprises no heteroatoms.

The phosphorous atom in formula (I) is directly bonded with a carbon atom in group L¹, so that the group containing the phosphorus atom can be understood as being a phosphonic acid or phosphonic acid ester group.

Preferably, the cross-linking monomer with at least one sulfur atom is representable by a structure of formula (Ia)

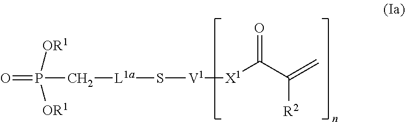

wherein the symbols n, V¹, X¹, R¹ and R² have the meaning described above in particular for formula (I), and

L^{1a} is a linear, branched or cyclic, saturated or unsaturated, aliphatic or heteroaliphatic group with 2 to 9 carbon atoms; an aromatic or heteroaromatic group with 4 to 12, preferably 5 to 10 carbon atoms or a group with 6 to 13 carbon atoms, which comprises at least one saturated or unsaturated, aliphatic and/or heteroaliphatic residue and at least one aromatic and/or heteroaromatic residue.

Preferred embodiments of group L^{1a} emerge from the specific embodiments of group L¹ described above, wherein the CH₂ group should, however, be taken into account.

The symbol R¹ in formula (I) or (Ia) is H or a hydrolyzable group. A hydrolyzable group is distinguished by the fact that it can be particularly easily transferred into an OH group with water or a corresponding reagent. Particularly easily means that the hydrolysis occurs faster than hydrolysis of the acrylic group. Preferably, the hydrolyzable group R¹ is an alkyl residue with 1 to 4, preferably 2 carbon atoms. Accordingly, the group R¹ preferably stands for hydrogen, methyl, ethyl, propyl or butyl, particularly preferred for hydrogen or ethyl.

Group V¹ is a saturated or unsaturated, aliphatic or heteroaliphatic group with 2 to 50, preferably 3 to 30 carbon atoms; an aromatic or heteroaromatic group with 5 to 50, preferably 6 to 30 carbon atoms or a group with 6 to 50, preferably 6 to 30 carbon atoms, which comprises at least one saturated or unsaturated, aliphatic and/or heteroaliphatic residue and at least one aromatic and/or heteroaromatic residue. Here, the definitions described above apply accordingly, wherein the number of carbon atoms can however be higher. Group V¹ has at least three bonding points. The precise number of bonding points is calculated from the number of polymerizable acrylic residues n, which is given in formula (I) or (Ia). The residue V¹ accordingly is (n+1) valent.

Preferably, group V¹ is a saturated or unsaturated, aliphatic or heteroaliphatic group with 2 to 50, preferably 3 to 30 carbon atoms or a group with 6 to 50, preferably 6 to 30 carbon atoms, which comprises at least one saturated or unsaturated, aliphatic and/or heteroaliphatic residue and at least one aromatic and/or heteroaromatic residue. Here, it is preferred that the sulfur atom in formula (I) and group X¹ in formula (I) are each bonded via an aliphatic carbon atom of group V¹, preferably a group —CH₂— or —CHR³—, wherein R³ has the meaning described for formula (I).

The index n stands for the number of acrylic groups, wherein this index is an integer in the range of 2 to 6, preferably 2 to 4, and particularly preferred 2 or 3.

The symbol X¹ in formula (I) or (Ia) is the same or different each time it occurs O or NR³, wherein R³ is H or an aliphatic, heteroaliphatic, aromatic or heteroaromatic group with 1 to 6 carbon atoms. Here, symbol X¹ in formula (I) or (Ia) preferably stands for O. Preferably, R³ stands for hydrogen, methyl, ethyl, propyl, butyl or phenyl. According to a particular embodiment, at least one X¹ can stand for NR³ and at least one X¹ can stand for O.

The symbol R² is H or an alkyl residue with 1 to 4 carbon atoms, preferably H or methyl and particularly preferred methyl.

The monomer with at least one sulfur atom according to the invention is distinguished by the fact that the sulfur atom in the structure according to formula (I) or (Ia) is separated from group X¹ by a maximum of 12 bonds, which group is spaced from the sulfur atom by the lowest number of bonds, wherein double bonds or triple bonds are counted as a single bond. Preferably, the sulfur atom in the structure according to formula (I) or (Ia) is separated from group X¹ by a maximum of 10, particularly preferred by a maximum of 8 bonds, which group is spaced from the sulfur atom by the lowest number of bonds, wherein double bonds or triple bonds are counted as a single bond.

Accordingly, a maximum of 11, preferably a maximum of 9 and specifically preferred a maximum of 7 covalent bonded atoms are located between the sulfur atom and the oxygen or nitrogen atom of group X¹. This value applies to the oxygen or nitrogen atom of group X¹, which has the lowest distance from the sulfur atom in the structure according to formula (I) or (Ia).

In a further embodiment, it can be provided that the sulfur atom in the structure according to formula (I) or (Ia) is separated from group X¹ by a maximum of 12 bonds, which group is spaced from the sulfur atom by the highest number of bonds, wherein double bonds or triple bonds are counted as a single bond. Preferably, the sulfur atom in the structure according to formula (I) or (Ia) is separated from group X¹ by a maximum of 10, particularly preferred by a maximum of 8 bonds, which group is spaced from the sulfur atom by the highest number of bonds, wherein double bonds or triple bonds are counted as a single bond.

Further, it can be provided that the phosphorous atom in the structure according to formula (I) or (Ia) is separated from group X¹ by a maximum of 20 bonds, which group is spaced from the sulfur atom by the highest number of bonds, wherein double bonds or triple bonds are counted as a single bond. Preferably, the phosphorous atom in the structure according to formula (I) or (Ia) is separated from group X¹ by a maximum of 18, particularly preferred by a maximum of 16 bonds, which group is spaced from the sulfur atom by the highest number of bonds, wherein double bonds or triple bonds are counted as a single bond.

Further, it can be provided that the phosphorous atom in the structure according to formula (I) or (Ia) is separated from group X¹ by at least 8 bonds, which group is spaced from the phosphorus atom by the lowest number of bonds, wherein double bonds or triple bonds are counted as a single bond. Preferably, the phosphorous atom in the structure according to formula (I) or (Ia) is separated from group X¹ by at least 10, particularly preferred by at least 11 bonds, which group is spaced from the sulfur atom by the lowest number of bonds, wherein double bonds or triple bonds are counted as a single bond.

Monomers according to the invention according to formula (I) or (Ia), in which the phosphorous or sulfur atom is separated from group X¹ by a low number of bonds, lead to improved polymer properties of curable compositions. Further, these compounds have a relatively low viscosity.

The expression “between the atoms” or “between the bonding points” means that the atoms or bonds are present in a main chain, which bond the two bonding points or the two atoms. The term “main chain” should be regarded as being different from a “side chain” or a substituent which tranches off from the main chain. In simplified terms, the number of bonds or atoms arranged “between the atoms” or “between the bonding points” does not change if a side group or a substituent is substituted with a hydrogen atom. If an atom in a main chain is substituted with one or two hydrogen atoms, this number changes.

Further, it can be provided that group V¹ in formula (I) or (Ia) comprises at least one aromatic and/or heteroaromatic residue, preferably at least one aromatic residue. This aromatic and/or heteroaromatic residue can be part of a linear, branched or cyclic, saturated or unsaturated, aliphatic or heteroaliphatic group, wherein the residue can in this case be a substituent of a CH₂ group or a group of formulae —(C═NR³)—, —C(═O)NR³—, —NR³—, P(═O)(R³), for example. Preferably, the aromatic and/or heteroaromatic residue of group V¹ can be a monocyclic residue, preferably a phenyl or phenylene residue, which if applicable can be substituted by one or more groups R³ wherein R³ has the meaning described above for formula (I).

Monomers according to the invention according to formula (I) or (Ia), in which group V¹ has at least one aromatic and/or heteroaromatic residue, lead to improved mechanical properties of hardened compositions.

Preferably, it can be provided that a monomer according to formula (I) or (Ia) has in total 1, 2 or 3 sulfur atoms, particularly preferred 1 or 2 sulfur atoms, and specifically preferred precisely one sulfur atom, wherein this number includes the sulfur atom shown in formula (I) or (Ia). If more than one sulfur atom is contained, this can be contained in group L¹ and/or group V¹.

In a particularly preferred manner, it can be provided that a monomer according to formula (I) or (Ia) has in total 1, 2 or 3 phosphorous atoms, particularly preferred 1 or 2 phosphorous atoms, and specifically preferred precisely one phosphorous atom, wherein this number includes the phosphorous atom shown in formula (I) or (Ia). If more than one phosphorous atom is contained, this can be contained in group L¹ and/or group V¹.

In a preferred embodiment, the monomer with at least one sulfur atom according to the invention can be representable by a structure (II)

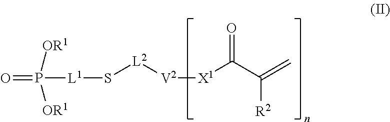

wherein the symbols n, L¹, X¹, R¹ and R² have the meaning described above in particular for formula (I), and

V² is a saturated or unsaturated, aliphatic and/or heteroaliphatic group with 2 to 10, preferably 3 to 6 carbon atoms; an aromatic or heteroaromatic group with 4 to 12, preferably 5 to 10 carbon atoms or a group with 6 to 14 carbon atoms, which comprises at least one saturated or unsaturated, aliphatic and/or heteroaliphatic residue and at least one aromatic and/or heteroaromatic residue; preferably a hydrocarbon residue with 3 to 5 carbon atoms; and

L² is a linear, branched or cyclic, saturated or unsaturated, aliphatic and/or heteroaliphatic ether or ester group with 2 to 30 carbon atoms; an aromatic and/or heteroaromatic ether or ester group with 5 to 30 carbon atoms or an ether or ester group with 6 to 30 carbon atoms, which comprises at least one saturated or unsaturated, aliphatic and/or heteroaliphatic residue and at least one aromatic and/or heteroaromatic residue.

The term ether group is known in the specialist field, wherein an ether group has at least one oxygen atom, which is bonded with two carbon atoms. Here, the carbon atom bonded with the oxygen atom can respectively be a component of a linear, branched or cyclic, saturated or unsaturated, aliphatic or heteroaliphatic group; an aromatic or heteroaromatic group or a group which comprises at least one saturated or unsaturated, aliphatic and/or heteroaliphatic residue and at least one aromatic and/or heteroaromatic residue. The number of carbon atoms results from the total number of both carbonaceous residues bonded with the oxygen atom of the ether group. However, it should also be taken into account that one of the carbon atoms with which the oxygen atom bonds can be a component of an adjacent group, such as group V² or group L³ described below in formula (III).

The term ester group is known to the specialist field, wherein an ester group is at least one group of the form —C(═O)O—, which is bonded with two carbon atoms. Here, the carbon atom bonded with the —C(═O)O— group can respectively be a component of a linear, branched or cyclic, saturated or unsaturated, aliphatic or heteroaliphatic group; an aromatic or heteroaromatic group, or a group comprising at least one saturated or unsaturated, aliphatic and/or heteroaliphatic residue and at least one aromatic and/or heteroaromatic residue. The number of carbon atoms results from the total number of both carbonaceous residues bonded with the —C(═O)O— group. However, it should also be taken into account that one of the carbon atoms with which the —C(═O)O— group bonds can be a component of an adjacent group, such as group V² or group L³ described in formula (III).

Preferably, it can be provided that a monomer according to formula (II) has a total of 1, 2 or 3 sulfur atoms, particularly preferred 1 or 2 sulfur atoms, and specifically preferred precisely one sulfur atom, wherein this number includes the sulfur atom shown in formula (II). If more than one sulfur atom is contained, this can be contained in group L¹, L² and/or V², preferably group L¹ and/or L².

Preferably, it can be provided that a monomer according to formula (II) has a total of 1, 2 or 3 phosphorous atoms, particularly preferred 1 or 2 phosphorous atoms, and specifically preferred precisely one phosphorous atom, wherein this number includes the phosphorous atom shown in formula (II). If more than one phosphorous atom is contained, this can be contained in group L¹, L² and/or V², preferably group L¹ and/or L².

In a preferred embodiment, the monomer with at least one sulfur atom according to the invention is representable by a structure (IIa)

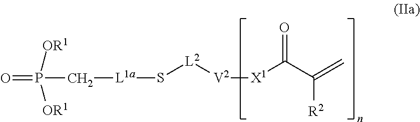

wherein the symbols n, L^{1a}, X¹, R¹ and R² have the meaning described for formula (Ia) and groups L² and V² have the meaning described for formula (II).

Preferably, it can be provided that group V² is selected from the following structures (V²-1), (V²-2) and/or (V²-3)

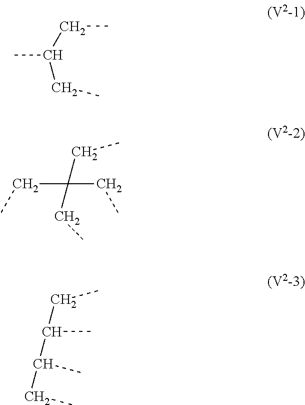

wherein the broken lines represent the bonds to groups L² or X¹.

Further, it can be provided that group L² is preferably representable by a structure of formula (L²-1)

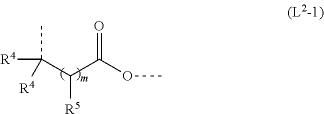

wherein each time it occurs, R⁴ is H, an alkyl group with 1 to 10 carbon atoms or an aromatic group with 6 to 8 carbon atoms, preferably H or a phenyl group; each time it occurs, R⁵ is independently H or an alkyl group with 1 to 10 carbon atoms, preferably H; the index m is an integer in the range of 0 to 8, preferably 0 to 7, and particularly preferred 0 to 5, and the broken lines represent the respective bond to the sulfur atom or the group V².

Further, it can be provided that the monomer with at least one sulfur atom is preferably representable by the structure (III)

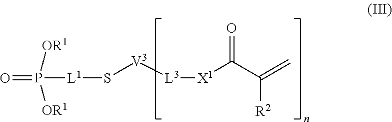

wherein the symbols n, L, X¹, R¹ and R² have the meaning described above in particular for formula (I),

V³ is a saturated or unsaturated, aliphatic and/or heteroaliphatic group with 2 to 10, preferably 3 to 6 carbon atoms; an aromatic and/or heteroaromatic group with 4 to 12, preferably 5 to 10 carbon atoms or a group with 6 to 14 carbon atoms, which comprises at least one saturated or unsaturated, aliphatic and/or heteroaliphatic residue and at least one aromatic and/or heteroaromatic residue; preferably an aliphatic group with 2 to 10, preferably 3 to 6 carbon atoms, in which one or more non-adjacent CH₂-Gruppen can be substituted with —R³C═CR³—, —C≡C—, —(C═O)—, —(C═S)—, —(C═NR³),—, —C(═O)O—, —C(═O)NR³—, —NR³—, P(═O)(R³), —[CH(CₓH₂ₓ)P(═O)(OR¹)₂]—, —O—, —S—, —(SO)— or (—SO₂)—, wherein R³ has the meaning described above for formula (I), and x is an integer in the range of 0 to 6, preferably 1 to 5; and

L³ is independently each time it occurs a linear, branched or cyclic, saturated or unsaturated, aliphatic and/or heteroaliphatic ether or ester group with 6 to 40, preferably 10 to 25 carbon atoms; an aromatic and/or heteroaromatic ether or ester group with 5 to 40, preferably 10 to 25 carbon atoms; or an ether or ester group with 6 to 40, preferably 10 to 25 carbon atoms, which comprises at least one saturated or unsaturated, aliphatic and/or heteroaliphatic residue and at least one aromatic and/or heteroaromatic residue; preferably an aliphatic and/or aromatic ether or ester group with 6 to 40, preferably 10 to 25 carbon atoms, in which one or more non-adjacent CH₂ groups can be substituted with —R³C═CR³—, —C≡C—, —(C═O)—, —(C═S)—, —(C═NR³),—, —C(═O)O—, —C(═O)NR³—, —NR³—, P(═O)(R³), —[CH(CₓH₂ₓ)P(═O)(OR¹)₂]—, —O—, —S—, —(SO)— or (—SO₂)—, wherein R³ has the meaning described above for formula (I), and x is an integer in the range of 0 to 6, preferably 1 to 5.

Preferably, it can be provided that a monomer according to formula (III) has a total of 1, 2 or 3 sulfur atoms, particularly preferred 1 or 2 sulfur atoms, and specifically preferred precisely one sulfur atom, wherein this number includes the sulfur atom shown in formula (III). If more than one sulfur atom is contained, this can be contained in group L¹, L³ and/or V³, preferably group L¹ and/or V³.

Preferably, it can be provided that a monomer according to formula (III) has a total of 1, 2 or 3 phosphorous atoms, particularly preferred 1 or 2 phosphorous atoms, and specifically preferred precisely one phosphorous atom, wherein this number includes the phosphorous atom shown in formula (III). If more than one phosphorous atom is contained, this can be contained in group L¹, L³ and/or V³, preferably group L¹ and/or V³.

Further, it can be provided that the monomer with at least one sulfur atom is preferably representable by structure (IIIa)

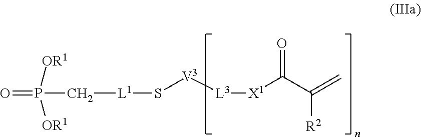

wherein the symbols n, L^{1a}, X¹, R¹ and R² have the meaning described above in particular for formula (Ia) and groups L³ and V³ have the meaning described above in particular for formula (III).

Further, it can be provided that group L³ is representable by a structure of formula (L³-1)

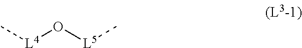

wherein each time it occurs, L⁴ and L⁵ independently are a linear, branched or cyclic alkylene group with 2 to 20, preferably 3 to 15 carbon atoms, in which one or more non-adjacent CH₂ groups can be substituted with —R³C═CR³—, —C≡C—, C═O, C═S, C═NR³, —C(═O)O—, —C(═O)NR³—, NR³, P(═O)(R³), —[CH(CₓH₂ₓ)P(═O)(OR¹)₂]—, —O—, —S—, SO or SO₂, wherein R¹ and R³ have the meaning described above for formula (I), and x is an integer in the range of 0 to 6, preferably 1 to 5, or an aryl or heteroaryl group with 5 to 20 carbon atoms, which can be substituted by one or more substituents R³; or a group with 6 to 40, preferably 10 to 25 carbon atoms, which comprises at least one saturated or unsaturated, aliphatic and/or heteroaliphatic residue and at least one aromatic and/or heteroaromatic residue; and the broken lines each represent the bond with group V³ or group X¹.

Preferably, it can be provided that the group L⁴ described in formula L³-1 is representable by a structure of formula (L⁴-1) or (L⁴-2)

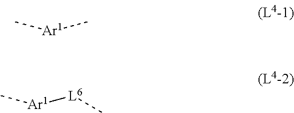

wherein Ar¹ an aryl or heteroaryl group with 4 to 12 carbon atoms, preferably 5 to 10 carbon atoms, preferably 6 carbon atoms, which can be substituted by one or more substituents R³; L⁶ is selected from C═O, C═S, C═NR³, —C(═O)O—, —C(═O)NR³—, NR³, P(═O)(R³), —O—, —S—, SO or SO₂ or a linear, branched or cyclic alkylene group with 1 to 6 carbon atoms, in which one or more non-adjacent CH₂-groups can be substituted with —R³C═CR³—, —C≡C—, C═O, C═S, C═NR³, —C(═O)O—, —C(═O)NR³—, NR³, P(═O)(R³), —[CH(CₓH₂ₓ)P(═O)(OR¹)₂]—, —O—, —S—, SO or SO₂, wherein R¹ and R³ have the meaning described in formula (I), x is an integer in the range of 0 to 6, preferably 1 to 5, and the broken lines each represent the bond with group V³ or the oxygen atom.

Preferably, group L⁵ is representable by a structure of formula (L⁵-1)

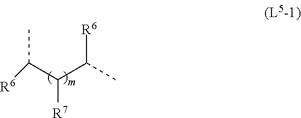

wherein each time it occurs, R⁶ is independently H, an alkyl group with 1 to 10 carbon atoms or an aromatic group with 6 to 8 carbon atoms, preferably H; R⁷ is H or an alkyl group with 1 to 10 carbon atoms, preferably H; and the index m is an integer in the range of 0 to 8, preferably 0 to 7, and particularly preferred 0 to 5, and the broken lines each represent the bond with the sulfur atom or group X¹.

Further, it can be provided that group V³ is representable by a structure of formulae (V³-1), (V³-2), (V³-3), (V³-4), (V³-5) or (V³-6)

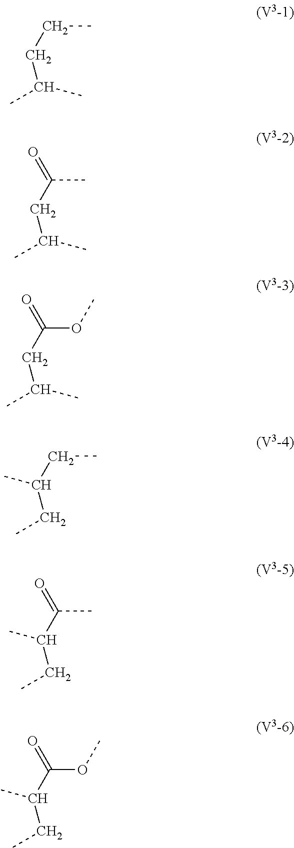

wherein the broken lines represent the bonds with S or groups L³.

Preferred monomers with at least one sulfur atom according to the present invention are representable, among others, by the formulae (IV), (V), (VI) and (VII)

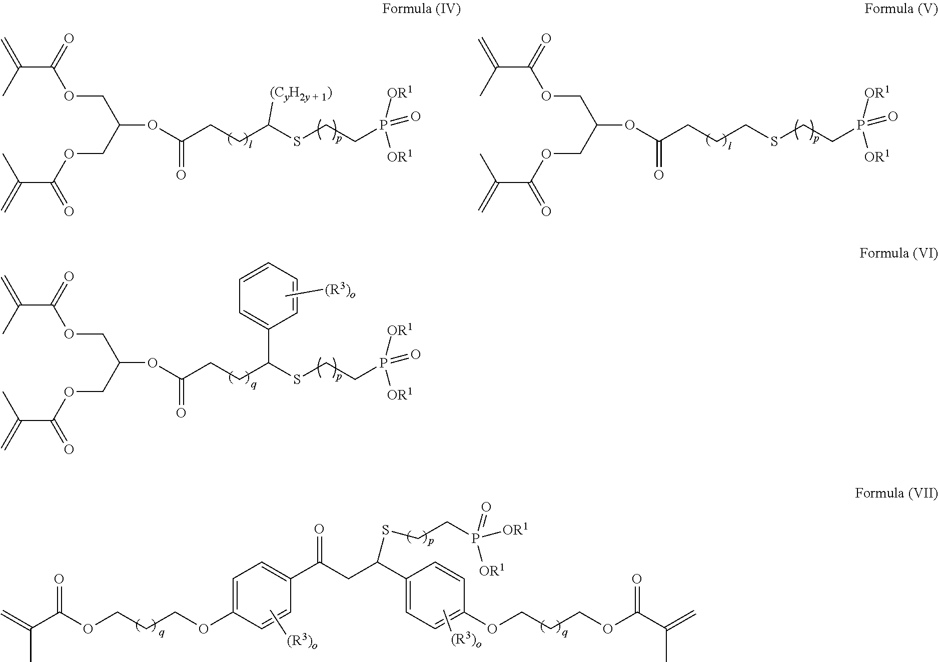

wherein the index I is 0, 1, 2, 3, 4, 5, 6 or 7, preferably 0, 1, 2, 3, 4, 5 or 6 and particularly preferred 0, 1, 2, 3, or 4; index o is 0, 1, 2, 3 or 4, preferably 0, 1 or 2, particularly preferred 0 or 1; index p is 1, 2, 3, 4, 5, 6 or 7, preferably 1, 2, 3, 4 or 5 and particularly preferred 2 or 3; index q is 1, 2, 3, 4, 5, 6 or 7, preferably 1, 2, 3, 4 or 5 and particularly preferred 1 or 2; and index y is 0, 1, 2, 3, 4, 5, 6 or 7, preferably 0, 1, 2, 3, 4, 5 or 6 and particularly preferred 0, 1, 2, 3, or 4, and the symbols R¹ and R³ have the meaning described for formula (I).

Particularly preferred monomers are representable by the formulae (IVa), (Va), (VIa) and (VIIa)

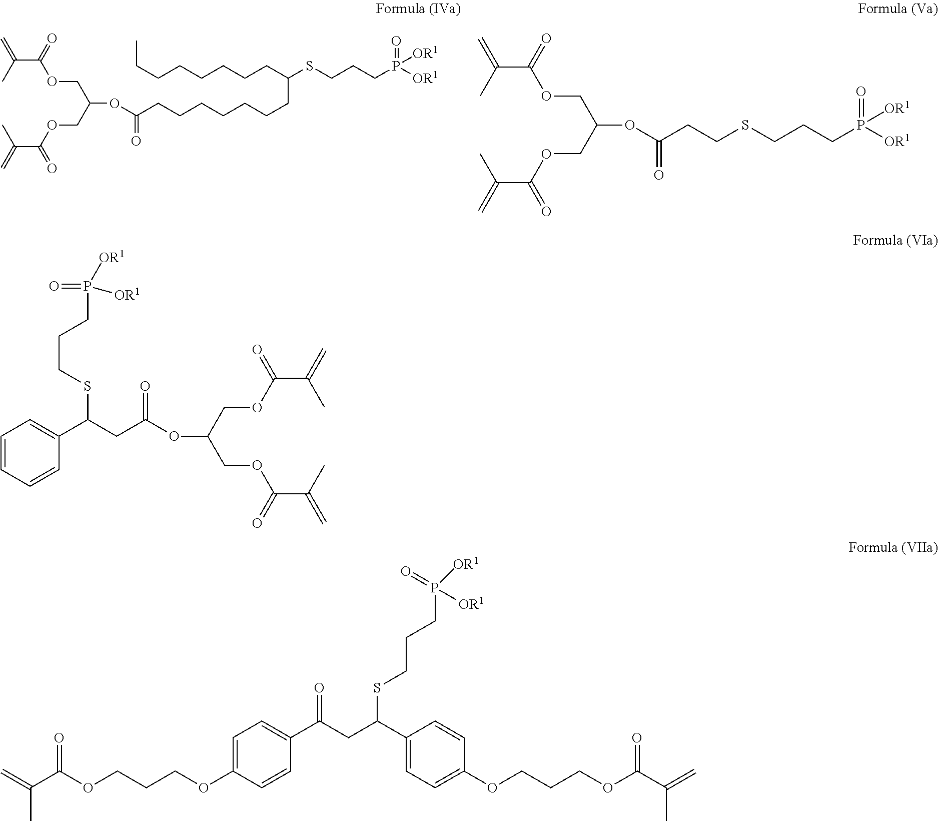

wherein the symbols R¹ have the meaning described for formula (I), wherein H is particularly preferred.

The monomers according to the invention can be produced from known compounds through a combination of method steps which are known per se. Thus, starting from a phosphonic acid ester derivative, which is converted with a thiocarboxylic acid compound to a thioester compound, a thiol with a phosphonic acid ester group can be obtained. The thiol with a phosphonic acid ester group can then be reacted, by a radical reaction with an unsaturated compound, to a thioether which either directly represents a compound according to the invention or is converted to a compound according to the invention in an esterification reaction. Here, a person skilled in the art will be provided with valuable information from the examples.

A preferred method for producing a monomer with at least one sulfur atom according to the invention is characterized in that an unsaturated compound is converted with a sulfurous compound according to formula (E-I)

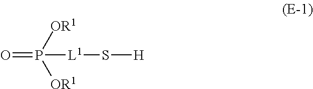

wherein the symbols L¹, and R¹ have the meaning described above in particular for formula (I).

A further subject of the present invention is a curable composition for use in dental medicine, comprising at least one monomer according to the invention.

A curable composition can, alongside the monomers with at least one sulfur atom according to the invention described in greater detail above, comprise at least one initiator for a radical polymerization of the monomers. Preferably, a curable composition can comprise monomers which are not representable by the formula (I) or a preferred embodiment of said formula. Further, a curable composition can contain organic or inorganic filler materials.

The preferred radically polymerizable monomers which are not representable by formula (I) or a preferred embodiment of said formula (co-monomers) include in particular mono- or polyfunctional (meth)acrylic acid derivatives. Monofunctional (meth)acrylic acid derivatives are understood as being compounds with one, polyfunctional (meth)acrylic acid derivatives are understood as being compounds with two or more, preferably 2 to 4 (meth)acrylic acid groups. Polyfunctional monomers have a cross-linking effect.

According to the invention, preferred mono- or polyfunctional (meth)acrylic acid derivatives are methyl-, ethyl-, hydroxyethyl-, butyl-, benzyl-, tetrahydrofurfuryl- or isobornyl (meth)acrylate, bisphenol-A-di(meth)acrylate, bis-GMA (an addition product from methacrylic acid and bisphenol-A-diglycidyl ether), UDMA (an addition product from 2-hydroxyethylmethacrylate (HEMA) and 2,2,4-trimethyl-hexamethylene diisocyanat (TMDI)), di-, tri- or tetraethylene glycoldi(meth)acrylate, trimethylolpropane tri(meth)acrylate, pentaerythrite tetra(meth)acrylate, glycerine di(meth)acrylate, glycerine tri(meth)acrylate, 1,4-butanediol di(meth)acrylate, 1,10-decanediol di(meth)acrylate and 1,12-dodecanediol di(meth)acrylate. The expression (meth)acrylate here stands for methacrylate, acrylate and mixtures of methacrylate and acrylate.

Particularly preferred mono- or polyfunctional (meth)acrylic acid derivatives are N-mono- or -disubstituted acrylamides such as N-ethyl acrylamide, N,N-dimethacrylamide, N-(2-hydroxyethyl) acrylamide or N-methyl-N-(2-hydroxyethyl) acrylamide, N monosubstituted methacrylamides such as N-ethyl methacrylamide or N-(2-hydroxyethyl) methacrylamide and N-vinylpyrrolidone and allyl ether. These monomers are distinguished by a very high hydrolysis stability and are suitable particularly as diluter monomers due to their relatively low viscosity.

Preferred polyfunctional (meth)acrylic acid derivatives with high hydrolysis stability are cross-linking pyrrolideones such as 1,6-bis(3-vinyl-2-pyrrolidonyl)-hexane, bisacrylamides such as methylene- or ethylene bisacrylamide and bis(meth)acrylamides such as N,N′-Diethyl-1, 3-bis(acrylamido)-propane, 1, 3-bis(methacrylamido)-propane, 1,4-bis (acrylamido)-butane or 1,4-bis(acryloyl)-piperazine, which can be synthesized with (meth)acrylic acid chloride through conversion from the corresponding diamines.

Preferably, mixtures of the above monomers are used.

The curable compositions according to the invention, which are particularly suitable as dental materials, can, alongside the polymerizable monomers with at least one sulfur atom of formula (I) and if applicable the above co-monomers, preferably also contain additional radically polymerizable monomers (adhesive monomers) which contain acid groups. Preferred acid groups are carboxylic acid groups, phosphonic acid groups, phosphoric acid groups and sulfonic acid groups.

Preferred polymerizable monomers with carboxylic acid groups are maleic acid, acrylic acid, methacrylic acid, 2-(hydroxymethyl) acrylic acid, 4-(meth)acryloyloxy-ethyltrimellitic acid hydride, 10-methacryloyloxydecyl malonic acid, N-(2-hydroxy-3-methacryloyloxypropyl)-N-phenylglycine and 4-vinylbenzoic acid. Preferred polymerizable monomers with phosphonic acid groups are vinyl phosphonic acid, 4-vinylphenyl phosphonic acid, 4-vinylbenzyl phosphonic acid, 2-methacryloyloxyethyl phosphonic acid, 2-methacrylamidoethyl phosphonic acid, 4-methacrylamido-4-methyl-pentyl phosphonic acid, 2-[4-(dihydroxyphosphoryl)-2-oxa-butyl]-acrylic acid, 2-[4-(dihydroxyphosphoryl)-2-oxa-butyl]-acrylic acid ethyl- and -2,4,6-trimethyl phenyl ester.

Preferred polymerizable monomers with phosphoric acid groups are 2-methacryloyloxypropyl mono- or -dihydrogen phosphate, 2-methacryloyloxyethyl mono- or -dihydrogen phosphate, 2-methacryloyloxyethylphenyl hydrogen phosphate, dipentaerythritol pentamethacryloyloxy phosphate, 10-methacryloyloxydecyl-di-hydrogen phosphate, phosphoric acid mono(1-acryloyl-piperidine-4-yl)-ester, 6-(methacrylamido)hexyl dihydrogen phosphate and 1,3-bis-(N-acryloyl-N-propyl-amino)-propane-2-yl-dihydrogen phosphate.

Preferred polymerizable monomers with sulfonic acid groups are vinyl sulfonic acid, 4-vinyl sulfonic acid and 3-(methacrylamido)propyl sulfonic acid.

Additionally, the curable compositions according to the invention, which are preferably suitable as dental materials, preferably also contain an initiator for the radical polymerization.

To initiate the radical polymerization, preferably benzophenone, benzoin and their derivatives or α-diketones or their derivatives, such as 9,10-phenanthrenequinone, 1-phenyl-propane-1,2-dione, diacetyl- or 4,4′-dichlorbenzil are used. In a particularly preferred manner, camphor quinone and 2,2-dimethoxy-2-phenyl-acetophenone and very particularly preferred, α-diketones in combination with amines such as 4-(kimethylamino)-benzoic acid ester, N,N-dimethylaminoethylmethacrylate, N,N-dimethyl-sym.-xylidine or triethanolamine are used as reduction agents. Particularly suited are also Norrish type-I photoinitiators, in particular acyl- or bisacyl phosphine oxides, monoacyltrialkyl- or diacyldialkyl germanium compounds such as benzoyltrimethylgermanium, dibenzoyldiethylgermanium or bis(4-methoxybenzoyl)diethylgermanium. Here, mixtures of the different photoinhibitors such as dibenzoyldiethylgermanium can be used in combination with campher chinone and 4-dimethylaminobenzoic acid ethyl ester.

As initiators for polymerization conducted at room temperature, preferably redox initiator combinations such as combinations of benzoyl peroxide with N,N-dimethyl-sym.-xylidine or N,N-dimethyl-p-toluidine are used. Furthermore, redox systems consisting of peroxidene or hydroperoxidine, and such reduction agents as e.g. ascorbic acid, barbiturates, thio urea derivatives or sulphinic acids are particularly suitable.

Furthermore, the compositions used according to the invention for improving the mechanical properties or for adjusting the viscosity can preferably also contain organic or inorganic filler material particles. Preferred inorganic particulate filler substances are amorphous spherical materials based on oxides such as ZrO₂ and TiO₂ or mixed oxides of SiO₂, ZrO₂ and/or TiO₂ with a medium average particle size of 0.005 to 2 μm, preferably 0.1 to 1 μm, nanoparticulate or micro-fine filler substances such as pyrogenic silicic acid or precipitation silicic acid with a medium average particle size of 5 to 200 nm, preferably 10 to 100 nm, mini-filler substances such as quartz, glass ceramic or glass powder with an average particle size of 0.01 to 10 μm, preferably 0.1 to 1 μm, and radiopaque filler substances such as ytterbium trifluoride or nanoparticulate tantalum (V)-oxide or barium sulfate with a medium average particle size of 10 to 1000 nm, preferably 100 to 300 nm.

The average particle sizes relate to the numerical average and can be determined using microscopic methods, such as scanning electron microscopy. With spherical particles, the sizes relate to the diameter, while with fibrous particles, they relate to the maximum extension of the respective particle.

Additionally, the compositions used according to the invention can contain further additives, above all solvents such as water, ethanol or acetone, or corresponding solvent agent mixtures, and e.g. stabilizers, aroma substances, dyes, microbiocidal active agents, fluoride ion emitting additives, optical brighteners, softeners or UV absorbers.

Particularly preferred are curable compositions which are preferably suitable as dental materials, on the basis of a polymerizable monomer with at least one sulfur atom with formula I and in particular on the basis of a polymerizable monomer with formula II or III, which contain the following components:

a) 0.1 to 50 weight %, in particular 1 to 40 weight %, preferably 2 to 30 weight %, and particularly preferred 5 to 20 weight % cross-linking monomer with at least one sulfur atom of formula I,

b) 0.01 to 10 weight %, preferably 0.1 to 3 weight % and particularly preferred 0.2 to 2 weight % initiator,

c) 0 to 80 weight %, preferably 1 to 60 weight % and particularly preferred 5 to 50 weight % co-monomer,

d) 0 to 30 weight %, preferably 0.5 to 15 weight % and particularly preferred 1 to 5 weight % adhesive monomer,

e) 0 to 80 weight % filler material, and

f) 0 to 70 weight %, preferably 0 to 60 weight % and particularly preferred 0 to 50 weight % solvent.

Here, the preferred filler material content is oriented to the desired application. Adhesives preferably contain 0 to 20 weight %, and cements and composites preferably contain 20 to 80 weight % filler material.

This also applies to the solvent content. Adhesives preferably contain 0 to 60 weight %, and particularly preferred 1 to 50 weight % solvent. Dental materials which contain water as a solvent are preferred. Particularly preferred are dental materials which contain 0 to 20 weight % and in particular 1 to 10 weight % water.

Further, the use of a monomer according to the invention or of a curable composition according to the present invention in dental medicine is the subject of the present invention.

The invention will now be explained in greater detail with reference to exemplary embodiments, without a restriction being intended as a result. Synthesis examples

The materials used were, as described below, obtained commercially and used without further purification:

allyl bromide (99%, Acros Organics), triethyl phosphite (98%, Aldrich Chemistry), thioacetic acid (98%, Acros Organics), triethylamine (99%, Acros Organics), 2,2′-azobis(2-methylpropionitrile) (AIBN, 98%, Sigma-Aldrich), trans-cinnamic acid (98+%, Acros Organics), 2,2-dimethoxy-2-phenylacetophenone (99%, Aldrich), oleic acid (Fluka), 3-brom-1-propanol (97%, Alfa Aesar), N,N′-dicyclohexylcarbodiimide (DCC, >99%, Fluka), N,N-dimethylpyridine-4-amine (DMAP, >98%, Fluka), bromtrimethyl silane (98%, Acros Organics), glycerine-1,3-dimethacrylate (isomer mixture, technical quality, 85%, Aldrich), 4-hydroxyacetophenone (99%, Alfa Aesar), 4-hydroxybenzaldehyde (99%, Acros Organics), N,N-dimethylacrylamide (99%, Acros Organics), dual-distilled water (Carl Roth), dichloromethane (Fisher Chemical), tetrahydrofurane (THF, p.a., VWR Chemicals), hydrochloric acid (37%, VWR Chemicals), toluol (p.a., Sigma Aldrich), phenothiazine (98%, Lancaster), methacryloyl chloride (97%, Alfa Aesar) and acryloyl chloride (97%, Fluka).

Diethyl(3-mercaptopropyl)phosphonate was obtained via methods known from the literature according to the following scheme:

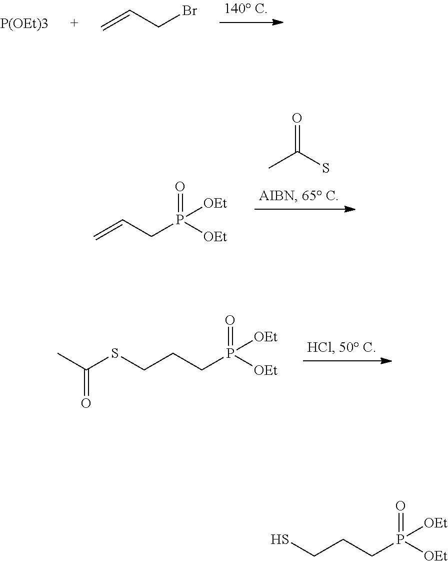

(Boutevin, B.; Hervaud, Y.; Mouledous, G.; Pelaprat, N. Phosphorus, Sulfur, and Silicon and the Related Elements 1998, 140, 125-133; Fourgeaud. Tetrahedron 2010, 66, 758; Putvinski, T.; Schilling, M. L.; Katz, H. E.; Chidsey, C. E.; Mujsce, A.; Emerson, A. Langmuir 1990, 6, 1567-1571)

The compounds obtained were characterized using the following methods:

¹H NMR measurements were conducted on a Bruker AVII-300 device at 300.13 MHz. The 5 scale relates to tetramethyl silane and was calibrated to a value of δ=7.26 ppm for dissolved CDCl₃. ¹³C NMR measurements were conducted at 75 MHz, ³¹P NMR measurements at 121 MHz.

Infrared (IR) spectra were recorded at room temperature with a Nicolet 6700 FT-IR spectrometer, which was equipped with an ATR unit. The measurements were conducted in the range of 4000 300 cm⁻¹.

A gas chromatograph (GC/MS (EI)) coupled with a mass spectrometer based on electron impact ionisation was used for analysis, wherein the measurement was conducted on a GC/MS system (“Triple quadrupole ion trap mass spectrometer” from Finnigan Trace DSQ and a Finnigan Trace GC Ultra). The device was calibrated to an m/z range of 4000 Da.

The purity of the compounds was examined with a mass spectrometer using an ion trap mass spectrometer (Finnigan LCQ Deca (Thermo Quest)). Ionisation was conducted with an electron spray.

### EXAMPLE 1

### Step 1

### Synthesis of 9-((3-diethoxyphosphoryl)thio)octadecane acid

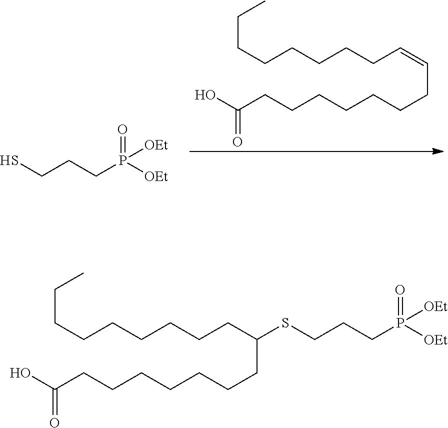 [SMILES: CCOP(=O)(CCCS)OCC ; CCCCCCCC/C=C\CCCCCCCC(=O)O ; CCCCCCCCCC(CCCCCCCC(=O)O)SCCCP(=O)(OCC)OCC]

6.5 g (23 mmol) oleic acid, 5.1 g (24 mmol) diethyl(3-mercaptopropyl) phosphonate and 295.8 mg (1.15 mmol) 2,2-dimethoxy-2-phenylacetophenone were mixed. The mixture was stirred for 6.5 hours under UV light. Educts that did not react were extracted through a 1-molar hydrochloric acid solution (3×). The raw product obtained was purified using column chromatography (ethyl acetate/methanol 95/5). A yellow liquid was obtained.

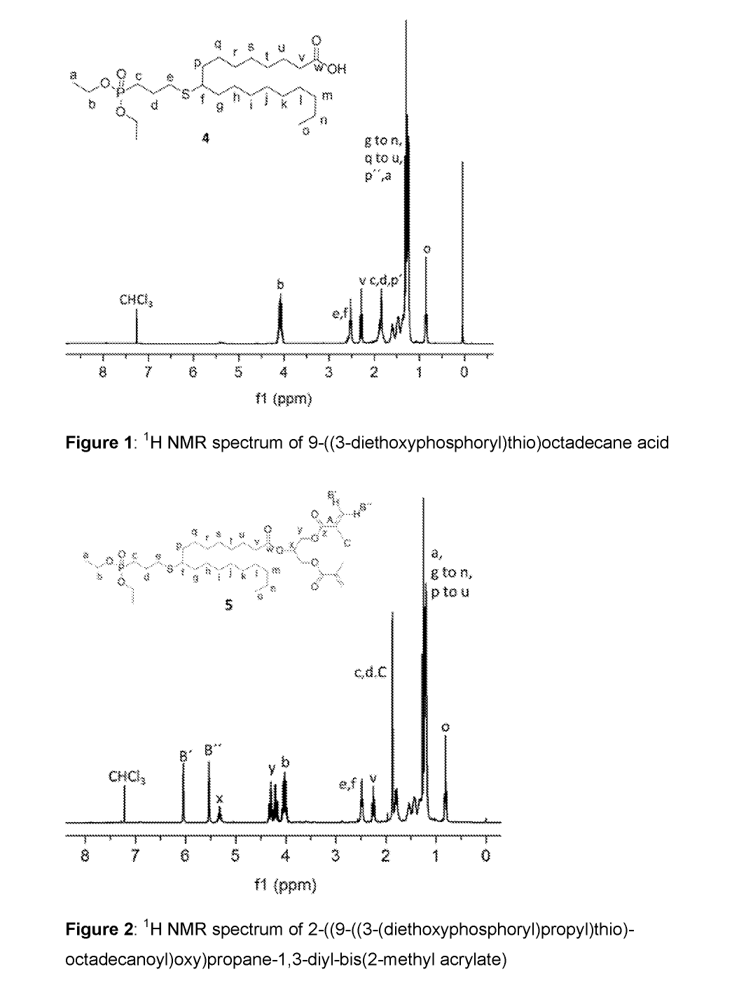

The ¹H NMR spectrum obtained of 9-((3-diethoxyphosphoryl)thio)octadecane acid is shown in FIG. 1.

Yield: 72%;

R_{f}-value: 0.35 (ethyl acetate/methanol 95/5);

FT-IR (diamond, cm⁻¹): \tilde{v}=3457 (b, v_{OH}), 2924/2853 (m, v_{C—H}), 1724 (w, v_{COOR}), 1456 (w, δ_{P C}), 1393, 1205 (m, v_{P—O}), 1097, 1055 (m, δ_{P—O—C}), 1024 (s, δ_{P—O—C}), 962, 784 (m, v_{P—O—C}), 723, 540, 494;

¹H-NMR (300 MHz, CDCl₃, δ [ppm]): 4.12-3.97 (m, 4H, b), 2.58-2.42 (m, 3H, e and f), 2.25 (t, ³J_{HH}=7.39 Hz, 2H, v), 1.91-1.71 (m, 5H, c, d and p′), 1.61-1.16 (m, 34H, g to n, q to u, p″ and a), 0.85-0.79 (m, 3H, o);

¹³C-NMR (75 MHz, CDCl₃, δ [ppm]): 176.68 (s, w), 60.79 (d, ²J_{PC}=6.56 Hz, b), 44.86 (s, f), 33.96-33.71 (d, p and g), 33.11 (s, v), 30.88 (s, m), 29.86 (d, ³J_{PC}=18.19 Hz, e), 28.65-27.92 (m, i to l and r to t), 25.83-25.57 (m, h and q), 23.79 (s, u), 23.67 (d, ¹J_{PC}=141.57 Hz, c), 21.82 (d, ²J_{PC}=4.57 Hz, d), 21.66 (s, n), 15.42 (s, ³J_{PC}=6.08 Hz, a), 13.09 (s, o);

³¹P-NMR (121 MHz, CDCl₃, δ [ppm]): 32.09 and 32.03 (2s, Markownikow and anti-Markownikow product);

MS (ESI) m/z: 495 [M+H⁺]; EAcalc. (%): C, 60.70. H, 10.39. S, 6.48; EAfound (%): C, 60.37. H: 10.14. S: 6.11.

### Step 2

### Synthesis of 2-((9-((3-(diethoxyphosphoryl)propyl)thio)octadecanoyl)oxy)propane-1,3-diyl-bis (2-methyl acrylate)

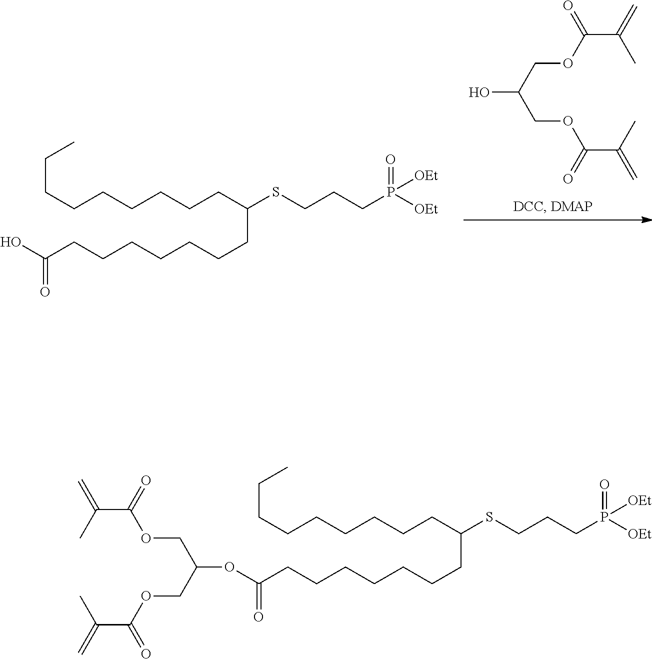 [SMILES: CCCCCCCCCC(CCCCCCCC(=O)O)SCCCP(=O)(OCC)OCC ; C=C(C)C(=O)OCC(O)COC(=O)C(=C)C ; C=C(C)C(=O)OCC(COC(=O)C(=C)C)OC(=O)CCCCCCCC(CCCCCCCCC)SCCCP(=O)(OCC)OCC]

In a 50 ml flask, 2 g (4 mmol) 9-((3-diethoxyphosphoryl)thio) octadecane acid were dissolved in 20 ml dry dichloromethane and cooled in an ice bath to 0° C. 0.83 (4 mmol) DCC and 50 mg (0.4 mmol) DMAP were suspended in 5 ml dry dichloromethane. The suspension was added drop by drop. After 2 hours, 0.9 ml (1 g, 4.4 mmol) glycerine-1,3-dimethacrylate were added; the mixture was brought to room temperature and stirred for 24 hours. Precipitated DCU was filtered and the filtrate was cooled with a dry ice-acetone mixture for 45 minutes (3×). The organic solution was concentrated under reduced pressure. The raw product was purified using column chromatography (silica gel) with ethyl acetate. A yellow liquid with a honey-like consistency was obtained.

The obtained ¹H NMR spectrum of 2-((9-((3-(diethoxyphosphoryl)propyl)thio)-octadecanoyl)oxy)propane-1,3-diyl-bis(2-methyl acrylate) is shown in FIG. 2.

Yield: 39%;

R_{f}-value: 0.66 (ethyl acetate);

FT-IR (diamond, cm⁻¹): \tilde{v}=2926/2854 (w, v_{C—H}), 1723 (s, v_{COOR}), 1638, 1454 (w, δ_{P—C}), 1321, 1294, 1235 (m, v_{P O}), 1154 (s, δ_{P—O—C}), 1097, 1057, 1027 (s, δ_{P—O—C}), 942, 812 (m, v_{P—O—C}), 723, 653, 541, 491;

¹H-NMR (300 MHz, CDCl₃, δ [ppm])=6.06-6.03 (m, 2H, B′), 5.55-5.51 (m, 2H, B″), 5.38-5.26 (m, 1H, x), 4.35-4.14 (m, 4H, y), 4.10-3.95 (m, 4H, b), 2.53-2.44 (m, 3H, e and f), 2.26 (td, ²J_{HH}=1.99 Hz, ³J_{HH}=7.51 Hz, 2H, v), 1.88-1.75 (m, 10H, c, d and C), 1.60-1.20 (m, 34H, a, g to n and p to u), 0.85-0.77 (m, 3H, o);

¹³C-NMR (75 MHz, CDCl₃, δ [ppm])=172.72 (s, w), 166.66 (s, z), 135.61 (s, A), 126.25 (s, B), 68.73 (s, x), 62.55 (s, y), 60.51 (d, ²J_{PC}=6.64 Hz, b), 45.79 (s, f), 34.79 (s, p and g), 34.04 (s, v), 31.82 (s, m), 30.82 (d, ³J_{PC}=17.94 Hz, e), 29.59-28.97 (m, i to l and r to t), 26.72 (s, h and q), 24.83 (s, u), 24.70 (d, ¹J_{PC}=141.55 Hz, c), 22.84 (d, ²J_{PC}=4.61 Hz, d), 22.60 (s, n), 18.17 (s, C), 16.40 (s, ³J_{PC}=6.05 Hz, a), 14.05 (s, o);

³¹P-NMR (121 MHz, CDCl₃, δ [ppm])=31.68 (s, 1P);

MS (ESI) m/z: 495 [C₂₅H₅₁O₅PS+H⁺], 706 [M+H⁺]; EA_{calc}. (%): C, 61, 34, H, 9.29, S, 4.55; EA_{found} (%): C, 61.17, H, 9.26, S, 4.12.

### EXAMPLE 2

### Synthesis of 3-((1-((1,3-bis(methacryloyloxy)propane-2-yl)oxy)-1-oxooctadecane-9-yl(thio)propyl)phosphonic acid

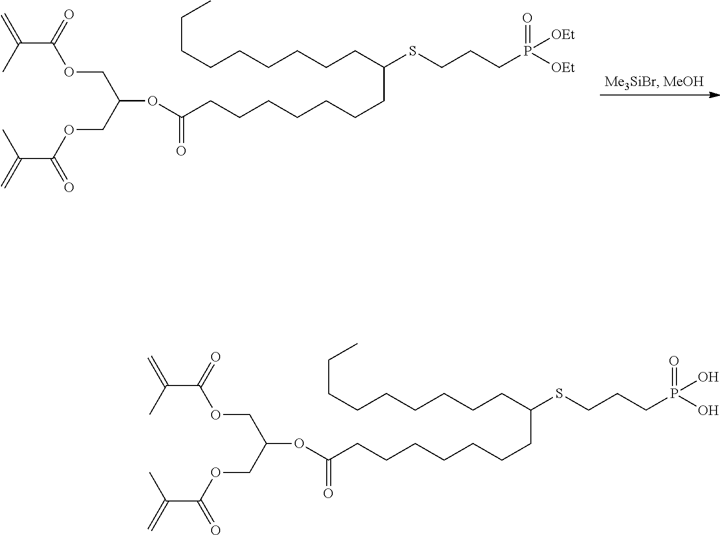 [SMILES: C=C(C)C(=O)OCC(COC(=O)C(=C)C)OC(=O)CCCCCCCC(CCCCCCCCC)SCCCP(=O)(OCC)OCC ; C=C(C)C(=O)OCC(COC(=O)C(=C)C)OC(=O)CCCCCCCC(CCCCCCCCC)SCCCP(=O)(O)O]

The hydrolysis of 2-((9-((3-(diethoxyphosphoryl)propyl)thio)octadecanoyl)oxy)-propane-1,3-diyl-bis(2-methyl acrylate) was conducted using the known method.

570 mg (0.81 mmol) 2-((9-((3-(diethoxyphosphoryl)propyl)thio)octadecanoyl)oxy)-propane-1,3-diyl bis 2-methyl acrylate was dissolved in 8.2 ml dry dichloromethane. The solution was de-gassed through the introduction of nitrogen for 10 minutes. Then, 1.07 μl (8.1 mmol) bromtrimethyl silane were added, and the solution was stirred for 9 hours with a bath temperature of 23° C. The organic solvent was removed under reduced pressure and dried under vacuum. The interim product was treated with 15 ml methanol and stirred for 1 hour at room temperature. Then, the solvent was removed at reduced pressure. After drying the product under vacuum, a yellow, highly viscous liquid was obtained.

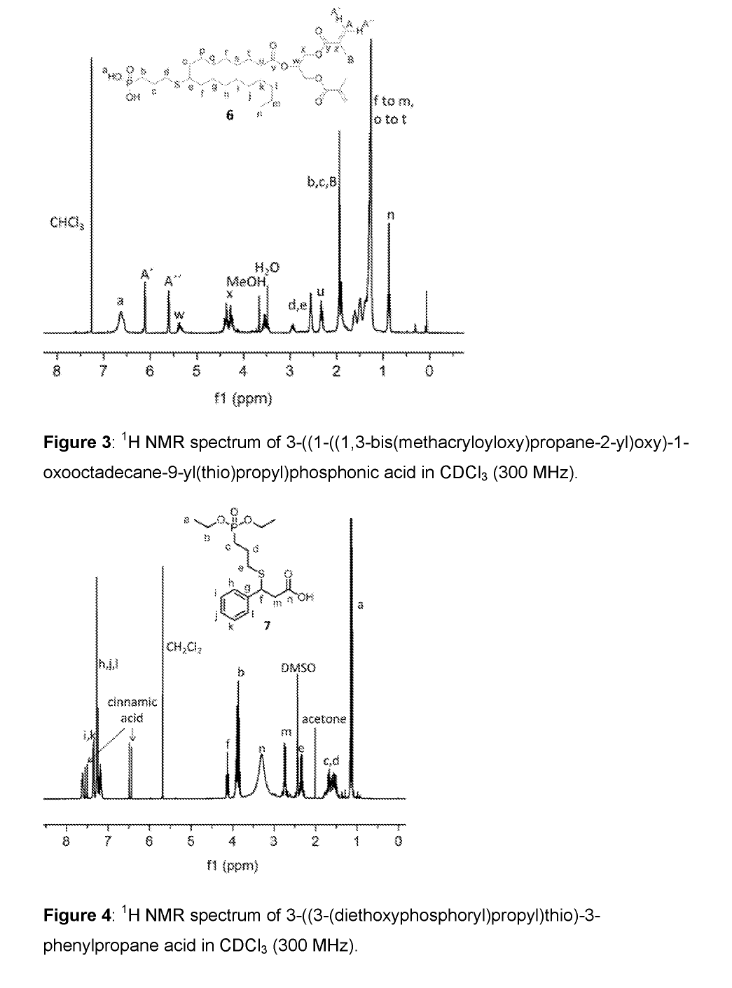

The obtained ¹H NMR spectrum of 3-((1-((1,3-bis(methacryloyloxy)propane-2-yl)oxy)-1-oxooctadecane-9-yl(thio)propyl)phosphonic acid is shown in FIG. 3.

Yield: 79% (0.64 mmol);

FT-IR (diamond, cm⁻¹): \tilde{v}=3386 (b, v_{O—H}), 2925/2854 (m, v_{C—H}), 1724 (s, v_{COOH}), 1638 (w, v_{CO═C}), 1455 (w, δ_{P—C}), 1378, 1294 (m, v_{P O}), 1154 (s, δ_{P—O—C}), 1007 (s, δ_{P—O—C}), 942, 813 (m, v_{P—O—C}), 723, 654, 520, 461;

¹H-NMR (300 MHz, CDCl₃, δ [pm])=6.56 (s, a), 6.07-6.04 (m, 2H, A′), 5.56-5.52 (m, 2H, A″), 5.36-5.22 (m, 1H, w), 4.41-4.11 (m, 4H, x), 2.53-2.44 (m, 3H, d and e), 2.26 (t, ²J_{HH}=7.22 Hz, 2H, u), 1.90-1.79 (m, 10H, b, c and B), 1.54-1.14 (m, 28H, f to m and o to t), 0.83-0.79 (m, 3H, n);

¹³C-NMR (75 MHz, CDCl₃, δ [ppm])=172.97 (s, v), 166.79 (s, y), 135.64 (s, z), 126.40 (s, A), 68.82 (s, w), 62.62 (s, x), 45.91 (s, e), 34.76 (s, o and f), 34.19 (s, u), 31.88 (s, l), 30.81 (d, ³J_{PC}=19.21 Hz, d), 29.68-29.03 (m, h to k and q to s), 26.74 (s, g and p), 24.90 (s, t), 24.89 (d, ¹J_{PC}=143.37 Hz, b), 22.67 (s, m), 22.53 (d, ²J_{PC}=4.17 Hz, c), 18.24 (s, B), 14.12 (s, n);

³¹P-NMR (121 MHz, CDCl₃, δ [ppm])=36.68 (s, 1P);

MS (ESI) m/z: 650 [M+H⁺], 678 [single saponified educt+H⁺], 729 [educt+Na⁺].

### EXAMPLE 3

### Step 1

### Synthesis of 3-((3-(diethoxyphosphoryl)propyl)thio)-3-phenyl propane acid

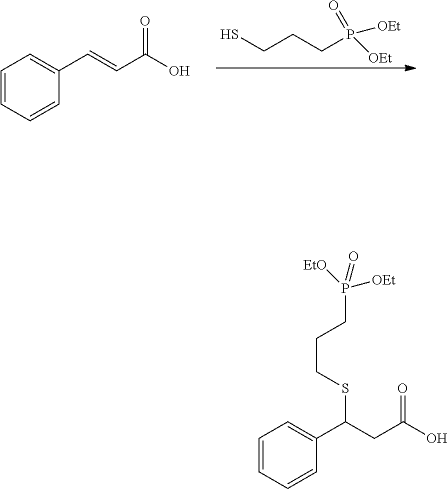 [SMILES: O=C(O)/C=C/c1ccccc1 ; CCOP(=O)(CCCS)OCC ; CCOP(=O)(CCCSC(CC(=O)O)c1ccccc1)OCC]

2 g (13.5 mmol) cinnamic acid and 2.56 g (12.1 mmol) diethyl (3-mercaptopropyl) phosphonate were dissolved in 6 ml THF. The solution was de-gassed through the introduction of nitrogen for 15 minutes. Following the addition of 0.4 ml (2.64 mmol) DBU (1,8-diazabicyclo[5.4.0]-7-undecene), the mixture was heated for approx. 48 hours under return flow. Following a cooling of the reaction mixture to room temperature, dichloromethane was added, and the mixture was washed twice with 1-molar, once with 0.1-molar hydrochloric acid, and with water. The solvent was removed under reduced pressure.

The obtained ¹H NMR spectrum of 3-((3-(diethoxyphosphoryl)propyl)thio)-3-phenyl propane acid is shown in FIG. 4. Raw yield: 2.09 g;

FT-IR (diamond, cm⁻¹): \tilde{v}=3465 (b, v_{OH}), 2981/2933/2905/2867 (w, v_{C—H}), 1707 (m, v_{COOH}), 1638 (w, v_{C═C}), 1575, 1494, 1451, 1393, 1366, 1306, 1261, 1190 (m, v_{C—OH}), 1097, 1020 (s, δ_{P—O—C}), 958 (s), 834, 796, 770/700 (m, δ_{benzyl}), 748, 699, 581, 530, 485;

¹H-NMR (300 MHz, DMSO-d6, δ [ppm]): 7.42-7.39 (m, 2H, i and k), 7.35-7.31 (m, 3H, h, j and l), 4.19 (t, ³J_{HH}=7.67 Hz, 1H, f), 4.02-3.87 (m, 4H, b), 3.36 (s, n), 2.81 (dd, ³J_{HH}=7.75 Hz, 2H, m), 2.47-2.33 (m, 2H, e), 1.89-1.50 (m, 4H, c and d), 1.20 (t, ³J_{HH}=7.07 Hz, 6H, a);

³¹P-NMR (121 MHz, DMSO-d6, δ [ppm]): 31.29 (s, 1P Edukt]), 31.10 (s, 1P, product);

MS (ESI) m/z: 361 [C₁₆H₂₅O₅PS+H⁺].

### Step 2

### Synthesis of 2-((3-((3-(diethoxyphosphoryl)propyl)thio-3-phenylpropanoyl)oxy)-propane-1,3-diyl bis (2-methyl acrylate)

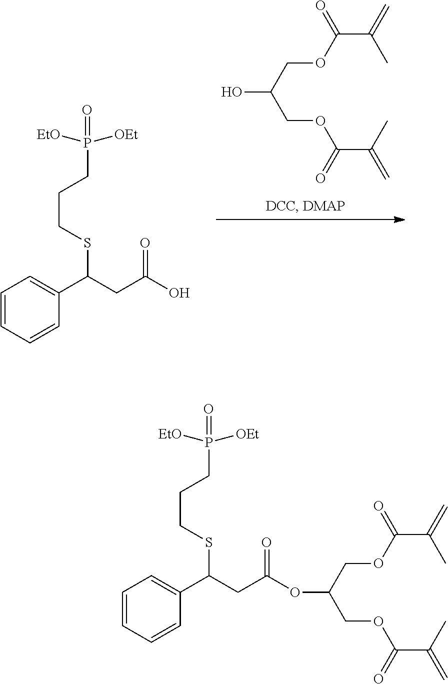 [SMILES: CCOP(=O)(CCCSC(CC(=O)O)c1ccccc1)OCC ; C=C(C)C(=O)OCC(O)COC(=O)C(=C)C ; C=C(C)C(=O)OCC(COC(=O)C(=C)C)OC(=O)CC(SCCCP(=O)(OCC)OCC)c1ccccc1]

In a 100 ml flask, 1 g (approx. 2.3 mmol) 3-((3-(diethoxyphosphoryl)propyl)thio)-3-phenyl propane acid was dissolved in 11 ml dry dichloromethane and cooled in an ice bath to 00° C. 711.8 mg (3.5 mmol) DCC and 42 mg (0.35 mmol) DMAP were suspended in 2 ml dry dichloromethane. The suspension was added drop by drop via a syringe. After 1.5 hours, 774 μl (3.8 mmol) glycerine-1,3-dimethacrylate were added; the mixture was brought to room temperature and stirred for 48 hours. Precipitated DCU was filtered, and the filtrate was cooled with a dry ice-acetone mixture for 60 minutes (3×). The organic solution was concentrated under reduced pressure. The raw product was purified using column chromotography (silica gel) with ethyl acetate and light petroleum (v:v 1:1). A light yellow liquid with a honey-like consistency was obtained.

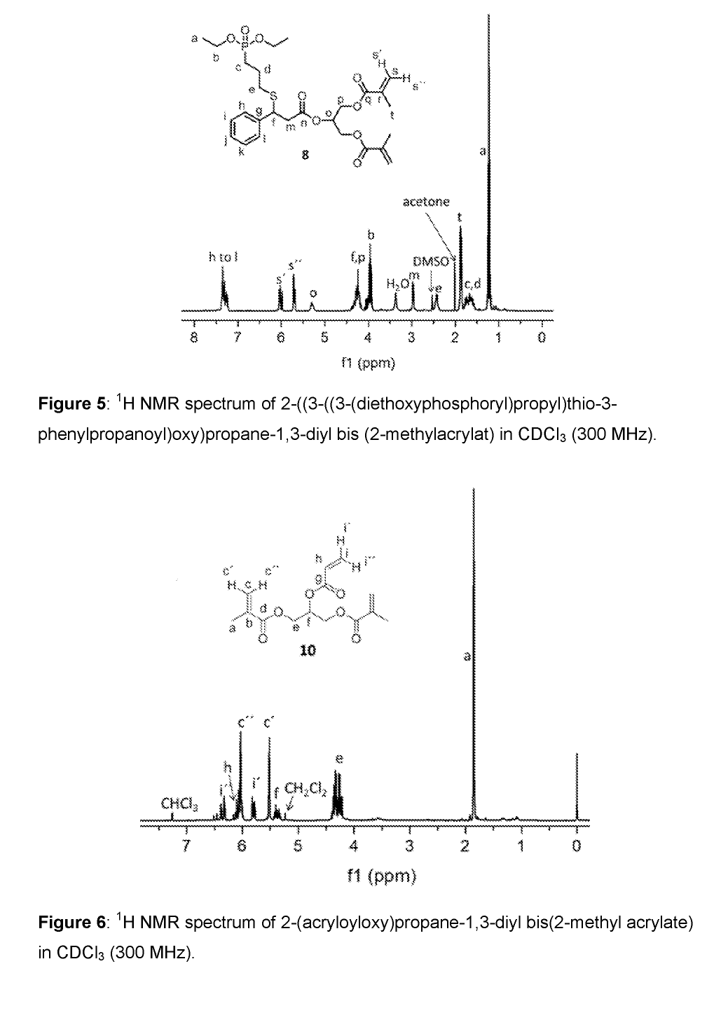

The obtained ¹H NMR spectrum of 2-((3-((3-(diethoxyphosphoryl)propyl)thio-3-phenylpropanoyl)oxy)propane-1,3-diyl bis (2-methyl acrylate) is shown in FIG. 5.

Yield: 53% (1.2 mmol);

R_{f}-value: 0.3 (ethyl acetate:light petroleum 1:1);

FT-IR (diamond, cm⁻¹): \tilde{v}=2961/2957/2928/2902 (w, v_{C—H}), 1720 (m, v_{C═O}), 1636 (w, v_{C═C}), 1453, 1377, 1321, 1294, 1234, (m, v_{P═O}), 1144, 1096, 1055, 1025 (s, δ_{P—O—C}), 950, 812/700 (m, (monosubstituted ring), 653, 532 cm⁻¹;

¹H-NMR (300 MHz, DMSO-d6, δ [ppm]): 7.36-7.18 (m, 5H, h to l), 6.03-5.95 (m, 2H, s′), 5.71-5.65 (m, 2H, s″), 5.32-5.20 (m, 1H, o), 4.37-4.14 (m, 5H, f and p), 4.06-3.87 (m, 4H, b), 2.95 (dd, ³J_{HH}=7.45 Hz, 2H, m), 2.46-2.32 (m, 2H, e), 1.88-1.82 (m, 6H, t), 1.79-1.52 (m, 4H, c and d), 1.20 (t, ³J_{HH}=7.08 Hz, 6H, a); 13C-NMR (75 MHz, DMSO-d6, δ [ppm]): 169.30 (s, n), 165.97 (s, q), 141.11 (s, g), 135.25 (s, r), 128.33-126.32 (quintet, h to l), 127.17 (s, s), 69.05 (s, o), 62.19 (d, ²J_{PC}=28.03 Hz, b), 60.79 (s, p), 44.25 (s, m), 40.36 (s, f), 30.74 (d, ³J_{PC}=17.55 Hz, e), 23.52 (d, ¹J_{PC}=138.96 Hz, c), 22.15 (d, ²J_{PC}=4.16 Hz, d), 17.18 (s, t), 16.19 (d, ³J_{PC}=5.8 Hz, a);

³¹P-NMR (121 MHz, DMSO-d6, δ [ppm]): 30.97 (s, 1P);

MS (ESI) m/z: 572 [C₂₇H₃₉O₉PS+H⁺]; EA_{calc}. (%): C, 56.83, H, 6.89, S, 5.62; EA_{found} (%): C, 56.43, H, 6.95, S, 5.54.

### EXAMPLE 4

### Synthesis of (3-((3-1,3-bis(methacryloyloxy)propane-2-yl)oxy-1-phenylpropyl)thio)propyl)-phosphonic acid

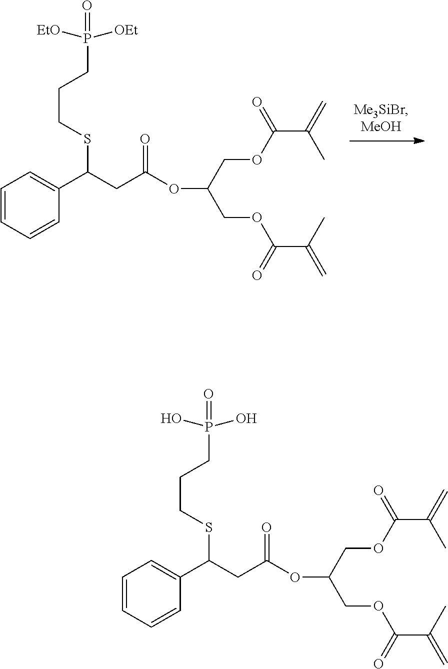 [SMILES: C=C(C)C(=O)OCC(COC(=O)C(=C)C)OC(=O)CC(SCCCP(=O)(OCC)OCC)c1ccccc1 ; C=C(C)C(=O)OCC(COC(=O)C(=C)C)OC(=O)CC(SCCCP(=O)(O)O)c1ccccc1]

According to the method described in example 2, 3-((3-1,3-bis(methacryloyloxy)propane-2-yl)oxy-1-phenylpropyl)thio)propyl)phosphonic acid was produced from 2-((3-((3-(diethoxyphosphoryl)propyl)thio-3-phenylpropanoyl)oxy)propane-1,3-diyl-bis-(2-methyl acrylate).

### EXAMPLE 5

### Step 1

### Synthesis of 2-(acryloyloxy)propane-1,3-diyl bis(2-methyl acrylate)

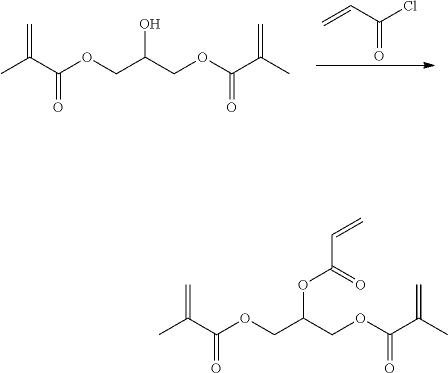

2-(acryloyloxy)propane-1,3-diyl bis(2-methyl acrylate) was obtained through the addition of 9.2 ml (10.3 g, 45 mmol) glycerine-1,3-dimethacrylate and 7.3 ml (5.3 g, 53 mmol) triethylamine to 100 ml dry dichloromethane in a 250-ml flask. The solution was cooled in an ice bath to 00° C. Then, 4.3 ml (4.8 g, 53 mmol) acrylic acide chloride, dissolved in 30 ml dry dichloromethane, were added drop by drop for 30 minutes. The solution was brought to room temperature and stirred for over 72 hours. The organic solution was washed three times with water, dried over MgSO₄ and filtered. Following the addition of phenothiazine, the solvent was removed under reduced pressure. A brown liquid was obtained.

The obtained ¹H NMR spectrum of 2-(acryloyloxy)propane-1,3-diyl bis(2-methyl acrylate) is shown in FIG. 6.

Yield: 83% (37.6 mmol);

FT-IR (diamond, cm⁻¹): \tilde{v}=2980/2960/2929 (w, v_{C—H}), 1719 (s, v_{COOR}), 1680, 1637 (w, v_{C═C}), 1453, 1406, 1321, 1293, 1264, 1149, 1050, 1066, 1043, 1012, 984 (m, δ_{C═C}), 943, 862, 809, 657, 598, 460;

¹H-NMR (300 MHz, CDCl₃, δ [ppm]): 6.35 (ddd, ²J_{HH}=5.10 Hz, ³J_{HH}=17.08 Hz, 1H, i″), 6.16-5.98 (m, 3H, h and c″), 5.80 (ddd, ²J_{HH}=3.92 Hz, ³J_{HH}=10.4 Hz, 1H, i′), 5.52 (s, 1H, c′), 5.44-5.31 (m, 1H, f), 4.40-4.19 (m, 4H, e), 1.85 (s, 6H, a);

¹³C-NMR (75 MHz, CDCl₃, δ [ppm]): 165.68 (s, d), 164.03 (s, g), 134.66 (s, b), 130.77 (s, i), 126.80 (s, h), 125.29 (s, c), 68.16 (s, f), 61.53 (s, e), 17.18 (s, a);

MS (ESI) m/z: 99 [C₅H₇O₂⁻], 197 [C₁₀H₁₃O₄⁻], 211 [C₁₁H₁₅O₄⁻], 214 [C₁₀H₁₄O₅], 283 [M+H⁺], 300 [M+NH₄⁺], 305 [M+Na⁺].

### Step 2

### Synthesis of 2-((3-((3-(diethoxyphosphoryl)propyl)thio)propanoyl)oxy)propane-1,3-diyl bis(2-methyl acrylate)

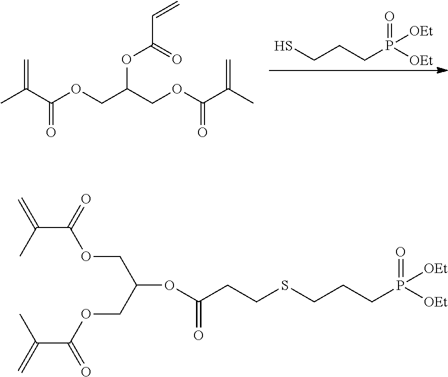 [SMILES: C=CC(=O)OC(COC(=O)C(=C)C)COC(=O)C(=C)C ; CCOP(=O)(CCCS)OCC ; C=C(C)C(=O)OCC(COC(=O)C(=C)C)OC(=O)CCSCCCP(=O)(OCC)OCC]

In a round flask, 5.1 g (18 mmol) 2-(acryloyloxy)propane-1,3-diyl bis(2-methyl acrylate), 3.1 g (14 mmol) diethyl (3-mercaptopropyl)phosphonate and 123 μl triethylamine were stirred for 6 days at room temperature. Then, 132 μl concentrated hydrochloric acid were added. The suspension was dissolved in dichloromethane and washed three times with water. The organic phase was dried over MgSO₄, filtered, and following the addition of phenothiazine, was concentrated under reduced pressure. The raw product was purified using column chromatography (silica gel) with ethyl acetate. A yellow-orange liquid with a honey-like consistency was obtained.

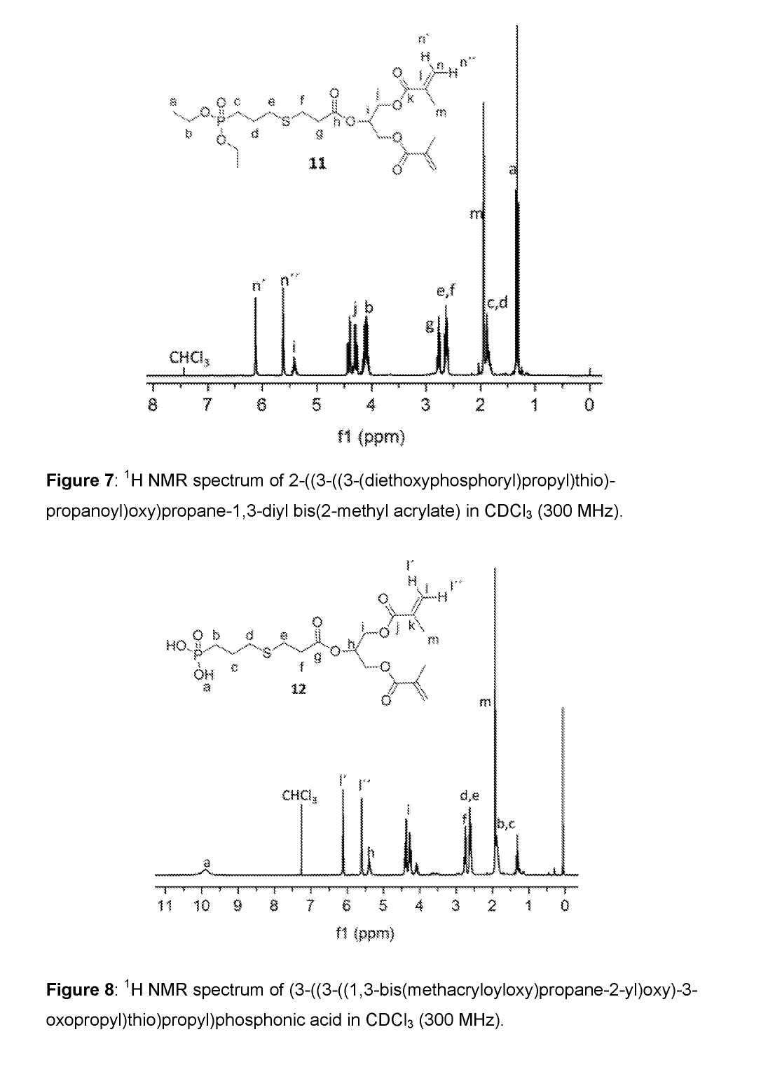

The obtained ¹H NMR spectrum of 2-((3-((3-(diethoxyphosphoryl)propyl)thio)-propanoyl)oxy)propane-1,3-diyl bis(2-methyl acrylate) is shown in FIG. 7.

Yield: 35% (5.1 mmol);

R_{f}-value: 0.39 (ethyl acetate);

FT-IR (diamond, cm⁻¹): \tilde{v}=2962/2929/2907 (w, v_{C—H}), 1722 (s, v_{COOR}), 1637 (w, v_{C═C}), 1452 (w, δ_{P C}), 1407, 1321, 1295, 1258, 1235 (w, v_{P—O}), 1156 (m, δ_{P—O—C}), 1022 (m, δ_{P—O—C}), 962, 863, 792 (m, v_{P—O—C}), 701, 661, 539, 480;

¹H-NMR (300 MHz, CDCl₃, δ [ppm]): 6.13-6.11 (m, 2H, n′), 5.63-5.60 (m, 2H, n″), 5.45-5.35 (m, 1H, i), 4.44-4.25 (m, 4H, j), 4.16-4.04 (m, 4H, b), 2.77 (t, ³J_{HH}=6.99 Hz, 2H, g), 2.63 (dd, ³J_{HH}=6.99 Hz, 4H, e and f), 1.95-1.79 (m, 10H, c, d and m), 1.33 (t, ³J_{HH}=7.13 Hz, 6H, a);

¹³C-NMR (75 MHz, CDCl3, δ [ppm]): 169.85 (s, h), 165.68 (s, k), 134.63 (s, l), 125.4 (s, n), 68.42 (s, i), 61.46 (s, j), 60.55 (d, ²J_{PC}=6.40 Hz, b), 33.68 (s, g), 31.52 (d, ³J_{PC}=18.56 Hz, e), 25.59 (s, f), 23.53 (d, ¹J_{PC}=141.68 Hz, c), 21.47 (d, ²J_{PC}=4.57 Hz, d), 17.24 (s, m), 15.47 (d, ³J_{PC}=6 Hz, a);

³¹P-NMR (121 MHz, CDCl₃, δ [ppm]): 31.23 (s, 1P);

MS (ESI) m/z: 495 [M+H⁺], 517 [M+Na⁺], 533 [C₂₁H₃₅O₉PS+K^{+]}; EA_{calc}. (%): C, 51.00, H, 7.13, S, 6.48; EA_{found} (%): C, 50.43, H, 7.22, S, 6.65.

### EXAMPLE 5

### Synthesis of (3-((3-((1,3-bis(methacryloyloxy)propane-2-yl)oxy)-3-oxopropyl)thio)-propyl) phosphonic acid

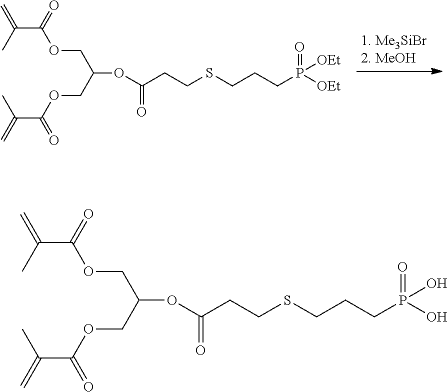 [SMILES: C=C(C)C(=O)OCC(COC(=O)C(=C)C)OC(=O)CCSCCCP(=O)(OCC)OCC ; C=C(C)C(=O)OCC(COC(=O)C(=C)C)OC(=O)CCSCCCP(=O)(O)O]

In a 100 ml double-neck flask, 2 g (4 mmol) 2-((3-((3-(diethoxy-phosphoryl)propyl)thio)propanoyl)oxy)propane-1,3-diyl bis(2-methyl acrylate) were dissolved in 5.3 ml dry dichloromethane. The solution was de-gassed through the introduction of nitrogen for 1 hour.

Then, 5.3 ml (40 mmol) bromtrimethyl silane were added, and the solution was stirred for 24 hours at a bath temperature of 23° C. The organic solvents were removed at reduced pressure and dried under vacuum. The interim product was treated with 5 ml methanol and stirred for 1 hour at room temperature. Then, the solvent was removed under reduced pressure. After the product was dried under vacuum, an olive-green, clear, highly viscous liquid was obtained.

The obtained ¹H NMR spectrum of (3-((3-((1,3-bis(methacryloyloxy)propane-2-yl)oxy)-3-oxopropyl)thio)propyl) phosphonic acid is shown in FIG. 8.

Yield: 90% (3.6 mmol);

FT-IR (diamond, cm⁻¹): \tilde{v}=3498 (b, v_{OH}), 2957/2921 (w, v_{C—H}), 1718 (m, v_{COOH}), 1637 (w, v_{C═C}), 1452 (w, δ_{P—C}), 1409, 1321, 1294, 1258, 1238 (w, v_{P—O}), 1151 (m, δ_{P—O—C}), 1006 (m, δ_{P—O—C}), 941, 813, 719, 651, 519, 456;

¹H-NMR (300 MHz, CDCl₃, δ [ppm]): 9.83 (s, a), 6.06-6.04 (m, 2H, l′), 5.56-5.52 (m, 2H, l″), 5.37-5.28 (m, 1H, h), 4.36-4.18 (m, 4H, i), 2.69 (t, ³J_{HH}=6.80 Hz, 2H, f), 2.58-2.52 (q, 4H, d and e), 1.89-1.71 (m, 10H, b, c and m);

¹³C-NMR (75 MHz, CDCl3, δ [ppm]): 171.14 (s, g), 166.83 (s, j), 135.56 (s, k), 126.59 (s, l), 69.46 (s, h), 62.47 (s, i), 34.66 (s, f), 32.31 (d, ³J_{PC}=18.72 Hz, d), 26.54 (s, e), 24.67 (d, ¹J_{PC}=144.55 Hz, b), 22.14 (d, ²J_{PC}=3.64 Hz, c), 18.23 (s, m);

³¹P-NMR (121 MHz, CDCl₃, δ [ppm]): 35.33 (s, 1P);

MS (ESI) m/z: 439 [M+H⁺], 462 [M+Na⁺].

### EXAMPLE 6

### Step 1

### Synthesis of 1,3-bis(4-(3-hydroxypropoxy)phenyl)prop-2-en-1-on

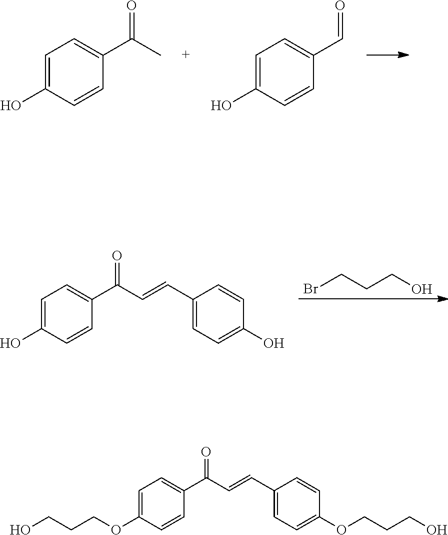

The chalcone 1,3-bis(4-hydroxyphenyl)prop-2-en-1-on was produced using a method known from the literature using a base-catalysed Claisen-Schmidt condensation of 4 hydroxybenzaldehyde and 4-hydroxyacetophenone (cf. N. Garg, T. Chandra, A. B. Jain and A. Kumar, European journal of medicinal chemistry 45:1529-1535 (2010)).

3.6 g (15 mmol) 1,3-bis(4-hydroxyphenyl)prop-2-en-1-on and 4.2 g (30 mmol) potassium carbonate were dissolved in 125 ml anhydrous acetone. Following the addition of a spatula tip of potassium iodide, 3.4 ml (5.28 g, 38 mmol) 3-brompropan-1-ol were added drop by drop via a dropping funnel. The mixture was heated for approx. 23 hours under return flow. The precipitation was filtered and washed with acetone. The clear, yellow filtrate was concentrated under reduced pressure and precipitated in water. The solid obtained was dissolved in acetone and again precipitated in water. Following filtration, the yellow solid was dried under vacuum.

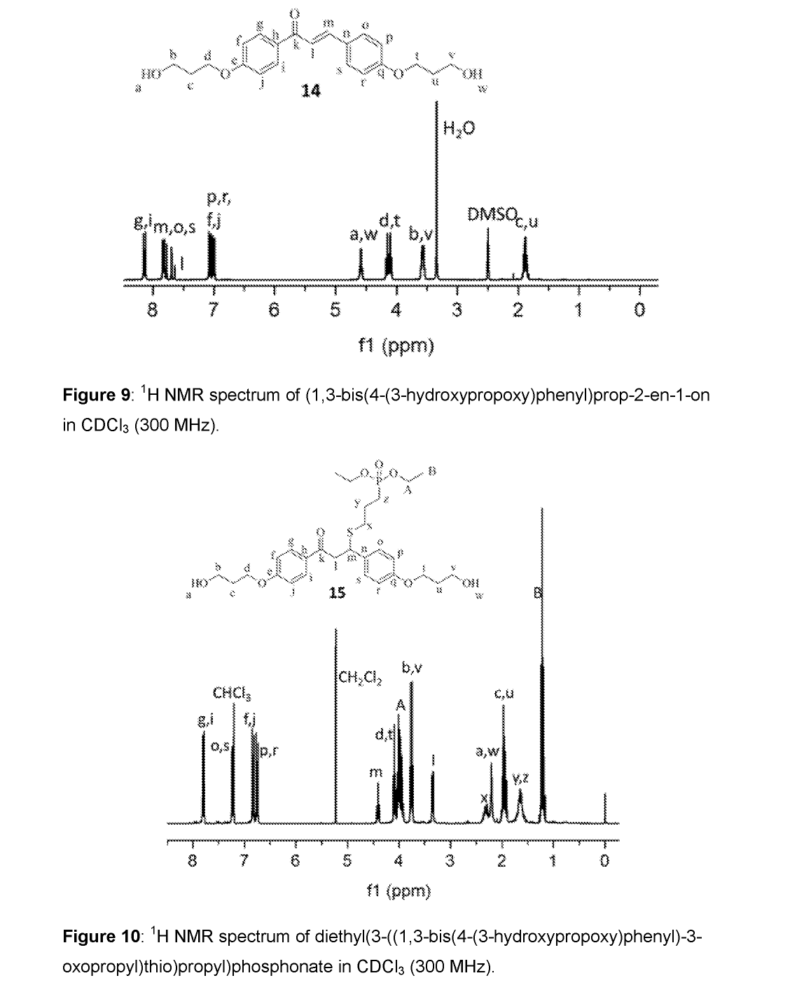

The obtained ¹H NMR spectrum of 1,3-bis(4-(3-hydroxypropoxy)phenyl)prop-2-en-1-on is shown in FIG. 9.

Yield: 37% (5.5 mmol);

FT-IR (diamond, cm⁻¹): \tilde{v}=3269 (b, v_{OH}), 2952/2935/2874 (w, v_{C—H}), 1661, 1627 (m, v_{C═O}), 1600/1572/1509 (m, v_{Benzyl}), 1470, 1421, 1388, 1334, 1305, 1293, 1233, 1174 (s, v_{C—OH}), 1116, 1079, 1033, 992, 944, 875, 862, 845, 833, 817 (s, (disubstituted ring), 756, 670, 648, 621, 597, 554, 527, 517;

¹H-NMR (300 MHz, DMSO-d6, δ [ppm]): 8.15 (d, ³J_{HH}=8.92 Hz, 2H, g and i), 7.85-7.78 (m, 3H, m, o and s), 7.68 (d, ³J_{HH}=15.53 Hz, 1H, l), 7.08 (d, 2H, p and r), 7.01 (d, 2H, f and j), 4.59 (q, ³J_{HH}=4.91 Hz, 2H, a and w), 4.18-4.09 (2t, ³J_{HH}=6.47 Hz, 4H, d and t), 3.61-3.54 (m, 4H, b and v), 1.94-1.84 (m, 4H, c and u);

¹³C-NMR (75 MHz, DMSO-d6, δ [ppm]): 178.16 (s, k), 162.51 (s, e), 160.63 (s, q), 143.07 (s, m), 130.74 (s, g and i), 130.61 (s, o and s), 130.48 (s, h), 127.27 (s, n), 119.34 (s, l), 114.76 (s, f and j), 114.31 (s, p and r), 64.87 (d, J=14.58 Hz, d and t), 57.14 (d, J=3.51 Hz, b and v), 31.96 (d, J=4.10 Hz, c and u);

MS (ESI) m/z: 357 [M+H⁺], 379 [M+K+].

### Step 2

### Synthesis of diethyl(3-((1,3-bis(4-(3-hydroxypropoxy)phenyl)-3-oxopropyl)thio)-propyl) phosphonate

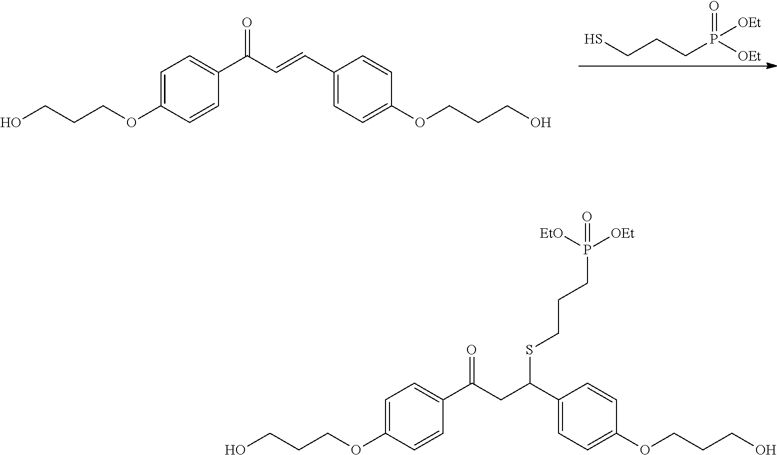 [SMILES: O=C(/C=C/c1ccc(OCCCO)cc1)c1ccc(OCCCO)cc1 ; CCOP(=O)(CCCS)OCC ; CCOP(=O)(CCCSC(CC(=O)c1ccc(OCCCO)cc1)c1ccc(OCCCO)cc1)OCC]

1 g (2.8 mmol) 1,3-bis(4-(3-hydroxypropoxy)phenyl)prop-2-en-1-on, 0.48 g (2.3 mmol) diethyl(3-mercaptopropyl) phosphonate and 38.8 μl (28.1 mg, 0.3 mmol) triethylamine were dissolved in 7 ml THF. The reaction mixture was purified through the introduction of nitrogen for 20 minutes, and was heated for 20 hours under return flow. Following a cooling of the reaction mixture to room temperature, 30 ml of dichloromethane were added and the mixture was washed twice with 1-molar, once with 0.1-molar hydrochloric acid and with water. The organic solvent was removed under reduced pressure. Non-converted educt was separated using column chromatography (silica gel) with ethyl acetate. The product was purified using column chromatography (silica gel) with chloroform:methanol (v:v 2:1).

The obtained ¹H NMR spectrum of diethyl(3-((1,3-bis(4-(3-hydroxypropoxy)phenyl)-3-oxopropyl)thio)propyl) phosphonate is shown in FIG. 10.

Yield: 75% (1.8 mmol);

R_{f}-value: 0.91 (chloroform:methanol 2:1);

FT-IR (diamond, cm⁻¹): \tilde{v}=3389 (b, v_{OH}), 2980/2932/2875 (w, v_{C—H}), 1737, 1672 (m, v_{C═O}), 1598/1574/1510 (m, v_{Benzyl}), 1472, 1421 (w, δ_{P—C}), 1392, 1334, 1304, 1248 (m, v_{P O}), 1226, 1169 (s, v_{C—OH}), 1097, 1053, 1023 (s, δ_{P—O—C}), 956, 831 (m, (disubstituted ring), 734, 699, 623, 543;

¹H-NMR (300 MHz, CDCl₃, δ [ppm]): 7.80 (d, ³J_{HH}=8.88 Hz, 2H, g and i), 7.23 (d, ³J_{HH}=8.72 Hz, 2H, o and s), 6.83 (d, ³J_{HH}=8.88 Hz, 2H, f and j), 6.76 (d, ³J_{HH}=8.72 Hz, 2H, p and r), 4.41 (t, ³J_{HH}=7.13 Hz, 1H, m), 4.09 (t, ³J_{HH}=6.09 Hz, 4H, d and t), 4.06-3.90 (m, 4H, A), 3.79-3.73 (2t, ³J_{HH}=5.76 Hz, 4H, b and v), 3.35 (dd, ³J_{HH}=7.08 Hz, 2H, l), 2.40-2.24 (m, 2H, x), 2.21 (s, 2H, a and w), 2.02-1.90 (m, 4H, c and u), 1.76-1.51 (m, 4H, y and z), 1.22 (t, ³J_{HH}=7.06 Hz, 6H, B);

¹³C-NMR (75 MHz, CDCl₃, δ [ppm]): 195.49 (s, k), 162.95 (s, e), 157.95 (s, q), 134.08 (s, n), 130.41 (s, g and i), 129.82 (s, h), 128.87 (s, o and s), 114.49 (s, f and j), 114.21 (s, p and r), 65.47 (d, J=3.56 Hz, d and t), 61.59 (d, J=6.47 Hz, b and v), 59.76 (d, ²J_{PC}=41.30 Hz, A), 45.02 (s, l), 44.01 (s, m), 32.02 (s, J=9.39 Hz, c and u), 31.77 (s, x), 24.53 (d, ¹J_{PC}=141.09 Hz, z), 22.19 (d, ²J_{PC}=4.23 Hz, y), 16.43 (d, ³J_{PC}=6.12 Hz, B);

³¹P-NMR (121 MHz, CDCl₃, δ [ppm]): 31.61 (s, 1P);

MS (ESI) m/z: 570 [M+H⁺]; EA_{calc}. (%): C, 59.14, H, 7.27, S, 5.64; EA_{found} (%): C, 59.28, H, 7.20, S, 5.29.

### Step 3

### Synthesis of methacrylated diethyl(3-((1,3-bis(4-(3-hydroxypropoxy)phenyl)-3-oxopropyl)thio)-propyl) phosphonate

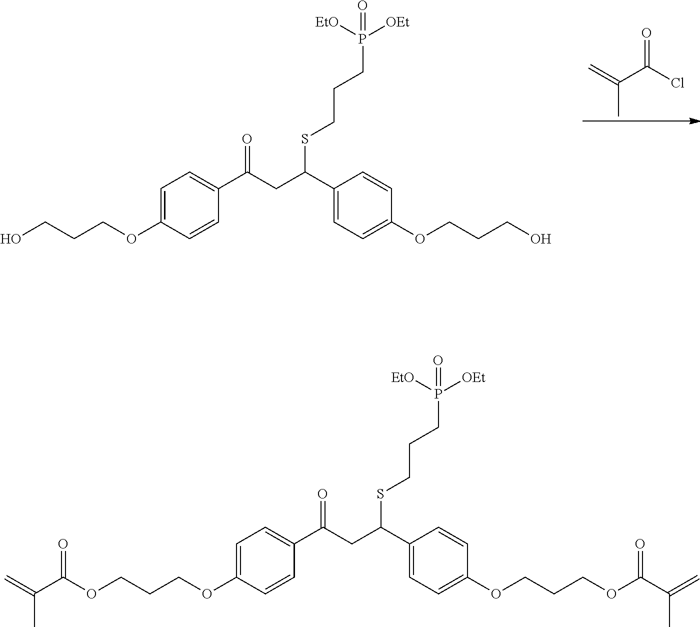

0.9 g (1.58 mmol) diethyl(3-((1,3-bis(4-(3-hydroxypropoxy)phenyl)-3-oxopropyl)thio)-propyl) phosphonate and 0.5 ml (0.37 g, 3.66 mmol) triethylamine were dissolved in 3.5 ml dry dichloromethane and cooled in an ice bath to 0° C. After an hour, 0.36 ml (0.38 g, 3.64 mmol) methacryl acid chloride, dissolved in 1 ml dry dichloromethane, were added drop by drop. The solution was brought to room temperature and stirred for 20 hours. The precipitation formed was filtered and the filtrate was washed three times with 50 ml water. The solvent was removed under reduced pressure. The raw product was purified using column chromatography (silica gel) with ethyl acetate. A light yellow liquid with a honey-like consistency was obtained.

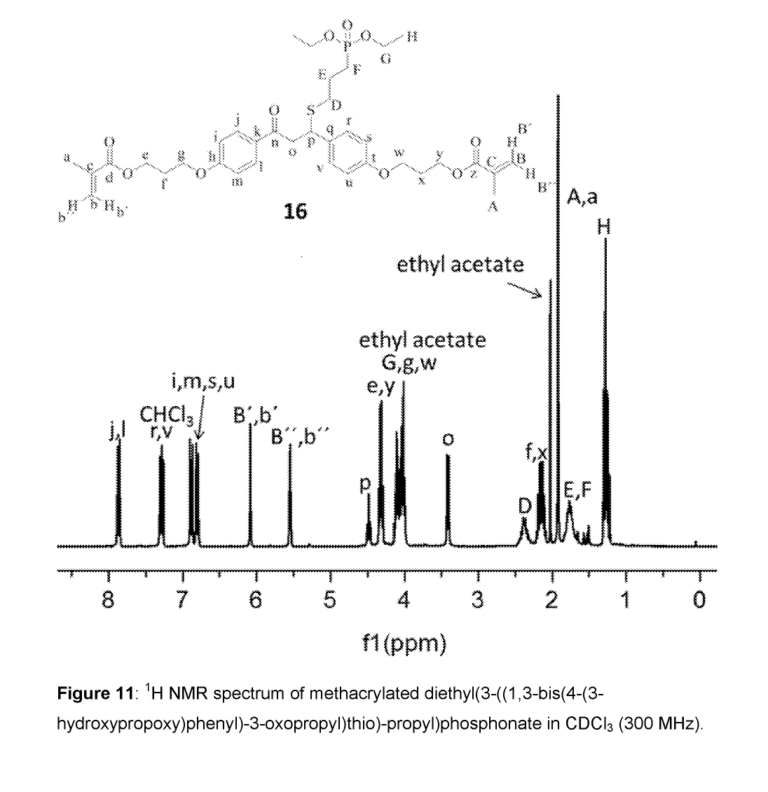

The obtained ¹H NMR spectrum of methacrylated diethyl(3-((1,3-bis(4-(3-hydroxypropoxy)phenyl)-3-oxopropyl)thio)-propyl) phosphonate is shown in FIG. 11.

Yield: 81% (1.3 mmol);

R_{f}-value: 0.47 (ethyl acetate);

FT-IR (diamond, cm⁻¹): \tilde{v}=2979/2930/2899/2872 (w, v_{C—H}), 1716 (m, v_{C═O}), 1676 (m, v_{C═C}), 1636, 1599/1575/1510 (m, v_{Benzyl}), 1472, 1452, 1421 (w, δ_{P—C}), 1319, 1296, 1232 (m, v_{P O}), 1162 (s, v_{C—OH}), 1114, 1047, 1026 (s, δ_{P—O—C}), 954, 831 (m, (disubstituted ring), 814, 650, 624, 543;

¹H-NMR (300 MHz, CDCl₃, δ [ppm]): 7.81 (d, ³J_{HH}=8.88 Hz, 2H, j and l), 7.24 (d, ³J_{HH}=8.65 Hz, 2H, r and v), 6.83 (d, ³J_{HH}=8.88 Hz, 2H, i and m), 6.75 (d, ³J_{HH}=8.65 Hz, 2H, s and u), 6.04-6.02 (m, 2H, b′ and B′), 5.50-5.47 (m, 2H, b″ and B″), 4.43 (t, ³J_{HH}=7.08 Hz, 1H; p), 4.26 (2t, ³J_{HH}=6.10 Hz, 4H, e and y), 4.08-3.94 (m, 8H, g, w, G), 3.35 (d, ³J_{HH}=7.08 Hz, 2H, o), 2.40-2.22 (m, 2H, D), 2.16-2.03 (m, 4H, f and x), 1.86 (s, 6H, a and A), 1.78-1.63 (m, 4H, E and F), 1.22 (t, ³J_{HH}=7.02 Hz, 6H, H);

¹³C-NMR (75 MHz, CDCl₃, δ [ppm]): 195.29 (s, n), 167.32 (s, d), 167.28 (s, z), 162.77 (s, h), 157.85 (s, t), 136.26 (s, c), 136.19 (s, C), 134.11 (s, q), 130.39 (s, j and l), 129.95 (s, k), 128.88 (s, r and v), 125.62 (s, b), 125.51 (s, B), 114.42 (s, i and m), 114.17 (s, s and u), 64.69 (s, g), 64.33 (s, w), 61.55 (s, e), 61.46 (s, y), 61.38 (d, ²J_{PC}=17.10 Hz, G), 45.08 (s, p), 43.75 (s, o), 31.90 (d, ³J_{PC}=18.02 Hz, D), 28.67 (s, f), 28.51 (s, x), 24.65 (d, ¹J_{PC}=141.03 Hz, F), 22.22 (d, ²J_{PC}=4.24 Hz, E), 18.31 (s, a and A), 16.45 (d, ³J_{PC}=5.81 Hz, H);

³¹P-NMR (121 MHz, CDCl₃, δ [ppm]): 31.46 (s, 1P);

MS (ESI) m/z: 705 [M+H⁺], EA_{calc}. (%): C, 61.35, H, 7.01, S, 4.55; EA_{found} (%): C, 60.65, H, 6.94, S, 4.63.

### EXAMPLE 7

### Synthesis of (3-((1,3-bis(4-3-8methacryloyloxy)propoxy)phenyl)-3-oxopropyl)thio)-propyl-phosphonic acid

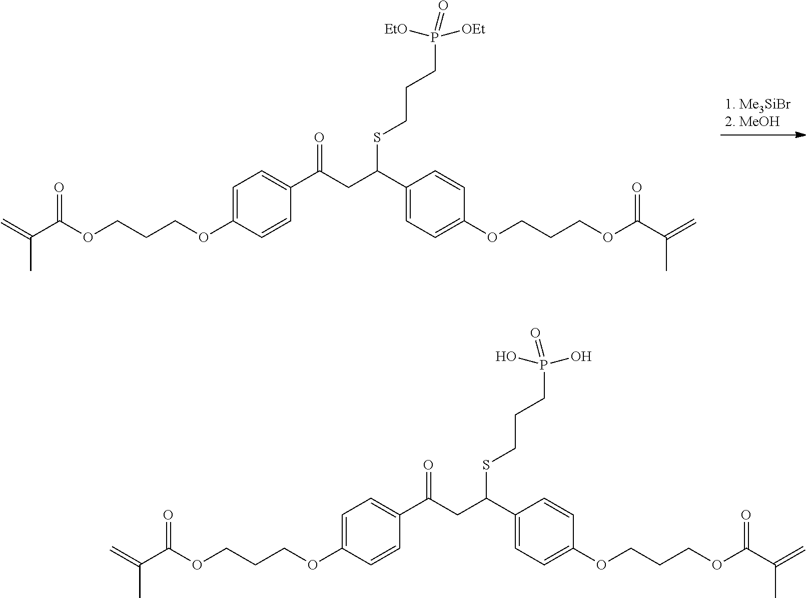

430 mg (0.61 mmol) methacrylated diethyl(3-((1,3-bis(4-(3-hydroxypropoxy)phenyl)-3-oxopropyl)thio)-propyl) phosphonate were dissolved in a 50 ml double-neck round flask in 6.2 ml dry dichloromethane. The solution was de-gassed through the introduction of nitrogen for 1 hour. Then, 805 μl (6.1 mmol) bromtrimethyl silane were added, and the solution was stirred for 9 hours at a bath temperature of 23° C. The organic solvent was removed under reduced pressure and dried under vacuum. The interim product was treated with 5 ml methanol and stirred for 30 minutes at room temperature. Then, the solvent was removed under reduced pressure. After drying the product under vacuum, a dark red, highly viscous liquid was obtained.

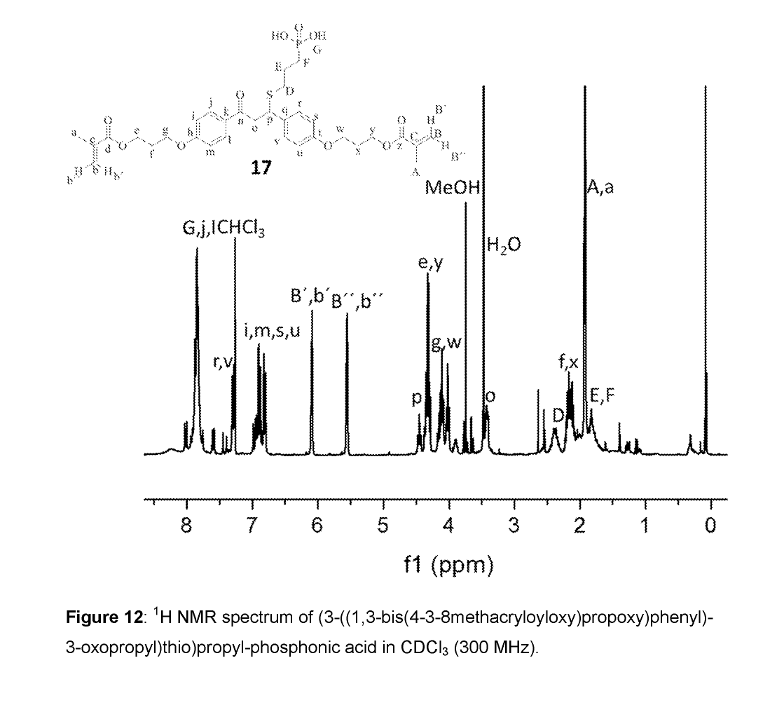

The obtained ¹H NMR spectrum of (3-((1,3-bis(4-3-8methacryloyloxy)-propoxy)phenyl)-3-oxopropyl)thio)propyl-phosphonic acid is shown in FIG. 12.

Yield: 89% (0-54 mmol);

FT-IR (diamond, cm⁻¹): \tilde{v}=3417 (b, v_{O—H}), 2961/2924/2867 (w, v_{C—H}), 1714 (m, v_{C═O}), 1676 (m, v_{C═C}), 1635, 1598/1572/1510 (m, v_{Benzyl}), 1471, 1451, 1422 (w, δ_{P—C}), 1356, 1298, 1257 (s, v_{P O}), 1166 (s, v_{C—OH}), 1112, 1043, 1011 (s, δ_{P—O—C}), 986, 827, 813 (s, (disubstituted ring), 734, 702, 648, 624, 621, 552, 543;

¹H-NMR (300 MHz, CDCl₃, δ [ppm]): 7.91-7.81 (m, 4H, j, l and G), 7.30 (d, ³J_{HH}=8.59 Hz, 2H, r and v), 6.8 (d, ³J_{HH}=8.93 Hz, 2H, i and m), 6.11-6.07 (m, 2H, s and u), 6.04-6.02 (m, 2H, b′ and B′), 5.57-5.53 (m, 2H, b″ and B″), 4.45 (t, ³J_{HH}=7.03 Hz, 1H, p), 4.38-4.29 (m, 4H, e and y), 4.17-4.00 (m, 4H, g and w), 3.42-3.40 (m, 2H, o), 2.45-2.32 (m, 2H, D), 2.22-2.08 (m, 4H, f and x), 1.94-1.89 (m, 6H, a and A), 1.85-1.68 (m, 4H, E and F);

¹³C-NMR (75 MHz, CDCl₃, δ [ppm]): 196.91 (s, n), 167.65 (s, d), 167.51 (s, z), 163.25 (s, h), 158.03 (s, t), 136.30 (s, c), 136.26 (s, C), 133.89 (s, q), 130.38 (s, j and l), 129.74 (s, k), 128.99 (s, r and v), 125.87 (s, b), 125.84 (s, B), 114.69 (s, i and m), 114.45 (s, s and u), 64.91 (s, g), 64.54 (s, w), 61.76 (s, e), 61.46 (s, y), 45.04 (s, p), 43.55 (s, o), 31.53 (d, ³J_{PC}=19.14 Hz, D), 28.74 (s, f), 28.60 (s, x), 24.55 (d, ¹J_{PC}=141.63 Hz, F), 21.61 (d, ²J_{PC}=2.80 Hz, E), 18.42 (s, a and A);

³¹P-NMR (121 MHz, CDCl₃, δ [ppm]): 35.46 (s, 1P);

MS (ESI) m/z: 581 [mono-methacrylated M+H⁺]; 649 [M+H⁺].

1. A cross-linking monomer with at least one sulfur atom, represented by a structure of formula (I) 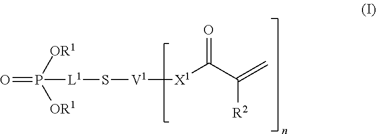
wherein the symbols have the following meaning:
L¹ is a linear, branched or cyclic, saturated or unsaturated, aliphatic or heteroaliphatic group with 2 to 10 carbon atoms; an aromatic or heteroaromatic group with 4 to 12 carbon atoms or a group with 6 to 14 carbon atoms, which comprises at least one saturated or unsaturated, aliphatic and/or heteroaliphatic residue and at least one aromatic and/or heteroaromatic residue, wherein the phosphorus atom of the group —P═O(OR¹)₂ is directly bonded with a carbon atom;
V¹ is a saturated or unsaturated, aliphatic or heteroaliphatic group with 2 to 50 carbon atoms; an aromatic or heteroaromatic group with 5 to 50 carbon atoms or a group with 6 to 50 carbon atoms, which comprises at least one saturated or unsaturated, aliphatic and/or heteroaliphatic residue and at least one aromatic and/or heteroaromatic residue;
X¹ is the same or different each time it occurs, either O or NR³;
R¹ is H or a hydrolyzable group;
R² is H or an alkyl residue with 1 to 4 carbon atoms;
R³ is H or an aliphatic, heteroaliphatic, aromatic or heteroaromatic group with 1 to 6 carbon atoms, and
n is 2, 3, 4, 5 or 6,
wherein the sulfur atom in the structure according to formula (I) is separated from group X¹ by a maximum of 12 bonds, which group is spaced from the sulfur atom by the lowest number of bonds, wherein double bonds or triple bonds are counted as a single bond.

2. The monomer according to claim 1, wherein group V¹ has at least one aromatic and/or heteroaromatic residue.

3. The monomer according to claim 2, wherein the aromatic and/or heteroaromatic residue of group V¹ is a monocyclic residue.

4. The monomer according to claim 1, wherein L¹ is a linear, branched, or cyclic alkylene group with 2 to 10 carbon atoms, in which one or more non-adjacent CH₂ groups is/are not substituted or is/are substituted with —R³C═CR³—, —C≡C—, —(C═O)—, —(C═S)—, —(C═NR³)—, —C(═O)O—, —C(═O)NR³—, —NR³—, P(═O)(R³), —[CH(CₓH₂ₓ)P(═O)(OR¹)₂]—, —O—, —S—, —(SO)—, or (—SO₂)—, wherein x is an integer in a range of 0 to 6.

5. The monomer according to claim 1, wherein the hydrolyzable group R¹ is an alkyl residue with 1 to 4, carbon atoms.

6. The monomer according to claim 1, wherein the monomer is represented by structure (II) 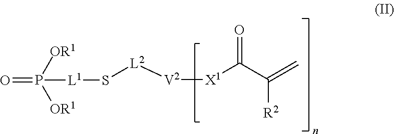
wherein
V² is a saturated or unsaturated, aliphatic and/or heteroaliphatic group with 2 to 10 carbon atoms; an aromatic or heteroaromatic group with 4 to 12 carbon atoms or a group with 6 to 14 carbon atoms, which comprises at least one saturated or unsaturated, aliphatic and/or heteroaliphatic residue and at least one aromatic and/or heteroaromatic residue; and
L² is a linear, branched or cyclic, saturated or unsaturated, aliphatic or heteroaliphatic ether or ester group with 2 to 30 carbon atoms; an aromatic and/or heteroaromatic ether or ester group with 5 to 30 carbon atoms or an ether or ester group with 6 to 30 carbon atoms, which comprises at least one saturated or unsaturated, aliphatic and/or heteroaliphatic residue and at least one aromatic and/or heteroaromatic residue.

7. The monomer according to claim 6, wherein group V² is selected from the following structures (V²-1), (V²-2) and/or (V²-3) 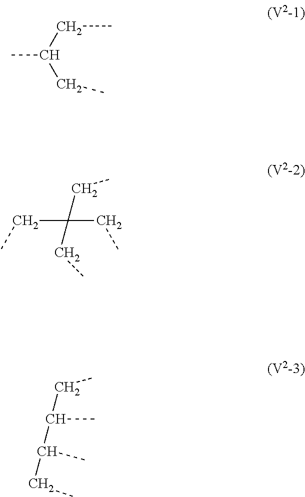
wherein the broken lines represent the bonds to groups L² or X¹.

8. The monomer according to claim 6, wherein group L² is represented by a structure of formula (L²-1) 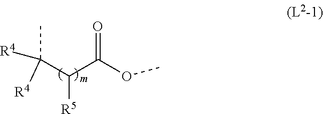
wherein, each time it occurs, R⁴ is H, an alkyl group with 1 to 10 carbon atoms or an aromatic group with 6 to 8 carbon atoms; each time it occurs, R⁵ is independently H or an alkyl group with 1 to 10 carbon atoms; the index m is an integer in the range of 0 to 8, and the broken lines each represent the bond to the sulfur atom or group V².

9. The monomer according to claim 1, wherein the monomer is represented by structure (III) 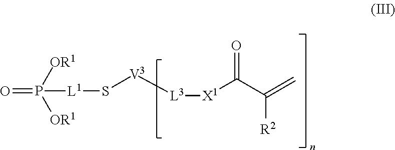
wherein
V³ is a saturated or unsaturated, aliphatic and/or heteroaliphatic group with 2 to 10 carbon atoms; an aromatic and/or heteroaromatic group with 4 to 12 carbon atoms or a group with 6 to 14 carbon atoms, which comprises at least one saturated or unsaturated, aliphatic and/or heteroaliphatic residue and at least one aromatic and/or heteroaromatic residue, in which one or more non-adjacent CH₂ groups is/are not substituted or is/are substituted with —R³C═CR³—, —C≡C—, —(C═O)—, —(C═S)—, —(C═NR³)—, —C(═O)O—, —C(═O)NR³—, —NR³—, P(═O)(R³), —[CH(CₓH₂ₓ)P(═O)(OR¹)₂]—, —O—, —S—, —(SO)—, or (—SO₂)—, wherein x is an integer in the range of 0 to 6; and
L³ is a linear, branched, or cyclic, saturated or unsaturated, aliphatic and/or heteroaliphatic ether or ester group with 6 to 40 carbon atoms; an aromatic and/or heteroaromatic ether or ester group with 6 to 40 carbon atoms; or an ether or ester group with 6 to 40 carbon atoms, which comprises at least one saturated or unsaturated, aliphatic and/or heteroaliphatic residue and at least one aromatic and/or heteroaromatic residue, in which one or more non-adjacent CH₂ groups is/are not substituted or is/are substituted with —R³C═CR³—, —C≡C—, —(C═O)—, —(C═S)—, —(C═NR³)—, —C(═O)O—, —C(═O)NR³—, —NR³—, P(═O)(R³), —[CH(CₓH₂ₓ)P(═O)(OR¹)₂]—, —O—, —S—, —(SO)—, or (—SO₂)—, wherein x is an integer in the range of 0 to 6.

10. The monomer according to claim 9, wherein group L³ is represented by a structure of formula (L³-1) 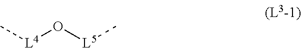
wherein, each time they occur, L⁴ and L⁵ independently are a linear, branched, or cyclical alkylene group with 2 to 20 carbon atoms, in which one or more non-adjacent CH₂ groups is/are not substituted or is/are substituted with —R³C═CR³—, —C≡C—, C═O, C═S, C═NR³, —C(═O)O—, —C(═O)NR³—, NR³, P(═O)(R³), —[CH(CₓH₂ₓ)P(═O)(OR¹)₂]—, —O—, —S—, SO, or SO₂, wherein x is an integer in the range of 0 to 6, or an aryl or heteroaryl group with 5 to 20 carbon atoms, which is not substituted or is substituted with one or more substituents R³; or a group with 6 to 40 carbon atoms, which comprises at least one saturated or unsaturated, aliphatic, and/or heteroaliphatic residue and at least one aromatic and/or heteroaromatic residue; and the broken lines each represent the bond to group V³ or group X¹.

11. The monomer according to claim 10, wherein group L⁴ is represented by a structure of formula (L⁴-1) or (L⁴-2) 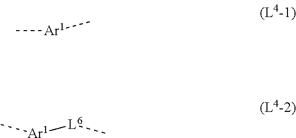
wherein Ar¹ is an aryl or heteroaryl group with 4 to 12 carbon atoms which is not substituted or is substituted with one or more substituents R³; L⁶ is selected from C═O, C═S, C═NR³, —C(═O)O—, —C(═O)NR³—, NR³, P(═O)(R³), —O—, —S—, SO, or SO₂ or a linear, branched, or cyclic alkylene group with 1 to 6 carbon atoms, in which one or more non-adjacent CH₂-groups is not substituted or is substituted with —R³C═CR³—, —C≡C—, C═O, C═S, C═NR³, —C(═O)O—, —C(═O)NR³—, NR³, P(═O)(R³), —[CH(CₓH₂ₓ)P(═O)(OR¹)₂]—, —O—, —S—, SO, or SO₂, wherein x is an integer in the range of 0 to 6 and the broken lines each represent the bond to group V³ or the oxygen atom.

12. The monomer according to claim 10, wherein group L⁵ is represented by a structure of formula (L⁵-1) 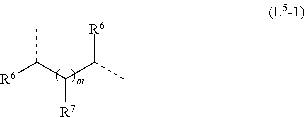
wherein, each time it occurs, R⁶ is independently H, an alkyl group with 1 to 10 carbon atoms or an aromatic group with 6 to 8 carbon atoms; R⁷ is H or an alkyl group with 1 to 10 carbon atoms; and the index m is an integer in the range of 0 to 8 and the broken lines represent bonds to the oxygen atom or the group X¹.

13. The monomer according to claim 9, wherein group V³ is represented by a structure of formulae (V³-1), (V³-2), (V³-3), (V³-4), (V³-5), or (V³-6) 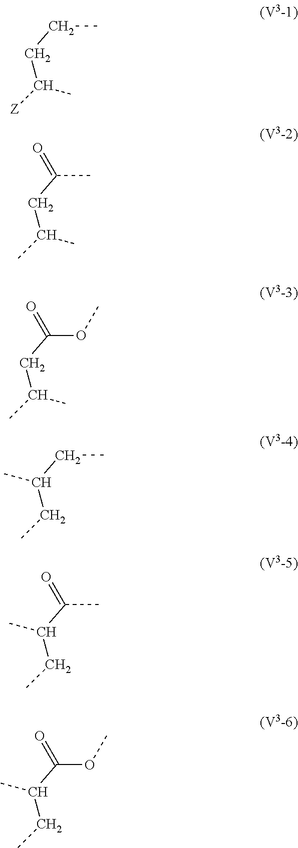
wherein the broken lines represent bonds to S or groups L³.

14. A curable composition for use in dental medicine, comprising at least one monomer according to claim 1.

15. A method for producing a monomer according claim 1, comprising reacting an unsaturated compound with a sulfurous compound in accordance with formula (E-I)

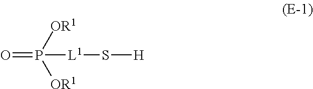

16. (canceled)

17. The monomer according to claim 2, wherein group V¹ has at least one aromatic residue.

18. The monomer according to claim 3, wherein the aromatic and/or heteroaromatic residue of group V¹ is a phenyl or phenylene residue.

19. The monomer according to claim 4, x is an integer in a range of 1 to 5.

20. The monomer according to claim 5, wherein the hydrolyzable group R¹ is an alkyl residue with 2, carbon atoms.

21. The monomer according to claim 6, wherein
V² is a saturated or unsaturated, aliphatic and/or heteroaliphatic group with 3 to 6 carbon atoms; an aromatic or heteroaromatic group with 4 to 12 carbon atoms or a group with 6 to 14 carbon atoms, which comprises at least one hydrocarbon residue with 3 to 5 carbon atoms.

22. The monomer according to claim 8, wherein, each time it occurs, R⁴ is H or a phenyl group; each time it occurs, R⁵ is independently H; and the index m is an integer in the range of 0 to 7.

23. The monomer according to claim 22, wherein the index m is an integer in the range of 0 to 5.

24. The monomer according to claim 9, wherein:
V³ is a saturated or unsaturated, aliphatic and/or heteroaliphatic group with 3 to 6 carbon atoms or an aliphatic group with 2 to 10 carbon atoms wherein and x is an integer in the range of 1 to 5; and
L³ is a linear, branched, or cyclic, saturated or unsaturated, aliphatic and/or heteroaliphatic ether or ester group with 10 to 25 carbon atoms; an aromatic and/or heteroaromatic ether or ester group with 10 to 25 carbon atoms; or an ether or ester group with 10 to 25 carbon atoms, which comprises at least one aliphatic and/or aromatic ether or ester group with 6 to 40, carbon atoms, wherein x is an integer in the range of 1 to 5.

25. The monomer according to claim 24, wherein:
V³ is a saturated or unsaturated, aliphatic and/or heteroaliphatic group with 3 to 6, carbon atoms; an aliphatic group with 3 to 6 carbon atoms; and
L³ is an ether or ester group with 10 to 25 carbon atoms, which comprises at least one aliphatic and/or aromatic ether or ester group with 10 to 25 carbon atoms.

26. The monomer according to claim 10, wherein, each time they occur, L⁴ and L⁵ independently are a linear, branched, or cyclical alkylene group with 3 to 15 carbon atoms, x is an integer in the range of 1 to 5, or a group with 10 to 25 carbon atoms, which comprises at least one saturated or unsaturated, aliphatic, and/or heteroaliphatic residue and at least one aromatic and/or heteroaromatic residue.

27. The monomer according to claim 11, wherein Ar¹ is an aryl or heteroaryl group with 6 carbon atoms and x is an integer in the range of 1 to 5.

28. The monomer according to claim 12, wherein, each time it occurs, R⁶ is independently H, R⁷ is H, and the index m is an integer in the range of 0 to 7.

29. The monomer according to claim 28, wherein the index m is an integer in the range of 0 to 5.
